(19) United States
(12) Patent Application Publication
(10) Pub. No.: US 2002/0165128 A1
(43) Pub. Date: Nov. 7, 2002

(54) Compositions and methods for proteolytically inactivating infectious agents using lactoferrin and related molecules

(76) Inventors: Andrew G. Plaut, Lexington, MA (US); Jiazhou Qiu, Medford, MA (US); Joseph W. St. Geme III, St. Louis, MO (US)

Correspondence Address: CLARK & ELBING LLP, 101 FEDERAL STREET, BOSTON, MA, 02110, US

(21) Appl. No.: 10/021,952

(22) Filed: Dec. 13, 2001

Related U.S. Application Data
(63) Continuation-in-part of application No. 09/289,997, filed on Apr. 12, 1999, now abandoned.
(60) Provisional application No. 60/081,564, filed on Apr. 13, 1998.

(51) Int. Cl. A61K 38/40

(52) U.S. Cl. 514/6

(57) ABSTRACT

A method for substantially reducing the pathogenicity of an infectious agent, without killing the infectious agent, by removing or degrading a surface protein of the infectious agent, by contacting the infectious agent with substantially pure, non-pasteurized, naturally occurring lactoferrin under conditions sufficient to remove or degrade the protein, is disclosed.

### CROSS REFERENCE TO RELATED APPLICATIONS

This application claims priority from U.S. provisional patent application Ser. No. 60/081,564 filed Apr. 13, 1998, and U.S. utility patent application Ser. No. 09/289,997 filed Apr. 12, 1999.

### STATEMENT AS TO FEDERALLY SPONSORED RESEARCH

The invention was made with funding from the National Institutes of Health, grants NIDDK DK34928, DE 09677, HD 20859, and AI 19641. The government has certain rights in the invention.

### BACKGROUND OF THE INVENTION

The invention relates to proteolytic compositions and methods for degrading proteins using lactoferrin and related molecules.

Infections, especially bacterial, fungal, and viral infections, are an increasingly serious health threat. The great variety of microbes and viruses, as well as their ability to develop resistance to the therapeutic agents used to inactivate them, presents a constant challenge in modern medicine. Relatively common infections can cause serious illness, and even death, in immunocompromised patients. Infections can also affect the long-term health of otherwise-healthy patients; even when the infections themselves are successfully treated, the secondary effects can cause lasting damage to the body.

The vast majority of people experience their first bacterial infection early in life. For example, an especially common early childhood infection is acute otitis media, which is a suppurative infection of the middle ear. By the time they have reached the age of three, 80% of children have suffered from acute otitis media, and 40-50% have experienced at least three episodes. Otitis media accounts for over one-third of all pediatric office visits in the United States and is the most common reason for prescribing oral antibiotics. Following each episode of otitis media, fluid persists in the middle ear for weeks to months, causing hearing impairment that can result in deficiencies in language acquisition, speech development, and cognitive achievement.

Most cases of otitis media are caused by infection with Streptococcus pneumoniae, Haemophilus influenzae, or Moraxella catarrhalis. Infection begins with colonization of the nasopharynx, followed by contiguous spread through the eustachian tube to the middle ear. Colonization is a complex process and involves the interplay of bacterial and host factors. Successful colonization requires that an organism evade local immune responses and overcome clearance by the mucociliary escalator. For example, both S. pneumoniae and H. influenzae secrete an IgA1 protease, which specifically cleaves and inactivates human IgA1, the predominant secretory antibody in the upper respiratory tract. In addition, all three of these respiratory pathogens elaborate adhesins, which promote attachment to the host epithelium and prevent the physical removal of bacteria from colonization sites.

### SUMMARY OF THE INVENTION

The present invention is based, in part, on the discovery that certain lactoferrin compositions have proteolytic activity. In one aspect, the invention features a method for substantially reducing the pathogenicity of an infectious agent, without killing the infectious agent, by removing or degrading a surface protein of the infectious agent; the method includes contacting the infectious agent with substantially pure, non-pasteurized, naturally occurring lactoferrin under conditions that are sufficient to remove or degrade the protein. Examples of infectious agents include bacteria, such as H. influenzae, and viruses. Examples of surface proteins include autotransported colonization factors, such as IgA1 protease, and adhesins, such as Hap.

In a related aspect, the invention features a method for substantially reducing the pathogenicity of an infectious agent, without killing the infectious agent, by removing or degrading a surface protein of the infectious agent; the method includes contacting the infectious agent with recombinant lactoferrin under conditions sufficient to remove or degrade the protein. In another related aspect, the invention features a method for substantially reducing the pathogenicity of an infectious agent, without killing the infectious agent, by removing or degrading a surface protein of the infectious agent; the method includes contacting the infectious agent with a substantially pure fragment of non-pasteurized, naturally occurring lactoferrin under conditions sufficient to remove or degrade the protein. A preferred fragment is the N-terminal lobe of lactoferrin.

In a second aspect, the invention features a method of inhibiting microbial colonization in a mammal, such as a human, by administering to the mammal a therapeutically effective amount of substantially pure, non-pasteurized, naturally-occurring lactoferrin. In a related aspect, the invention features a method of inhibiting microbial colonization in a mammal by administering to the mammal a therapeutically effective amount of a substantially pure fragment of non-pasteurized, naturally-occurring lactoferrin, such as the N-terminal lobe of lactoferrin.

In a third aspect, the invention features a method for substantially inactivating an infectious agent; the method includes contacting the infectious agent with substantially pure, non-pasteurized, naturally-occurring lactoferrin under inactivating conditions. In a related aspect, the invention features a method for substantially inactivating an infectious agent; the method includes contacting the infectious agent with a substantially pure fragment of lactoferrin under inactivating conditions, where the fragment has at least 100 amino acid residues. In a preferred method, the fragment has at least 200 amino acid residues. For example, a preferred fragment is the N-terminal lobe of lactoferrin. Preferably, the fragment is non-pasteurized and/or is isolated from naturally-occurring lactoferrin.

In a fourth aspect, the invention features an antimicrobial pharmaceutical composition including substantially pure, non-pasteurized, naturally-occurring lactoferrin and a pharmaceutically acceptable carrier. The composition may be formulated, e.g., for administration by the gastrointestinal tract (e.g., by oral administration), by inhalation, by the mucous membranes, or by the eyes. In a related aspect, the invention features an antimicrobial pharmaceutical composition including a substantially pure fragment of non-pasteurized, naturally-occurring lactoferrin and a pharmaceutically acceptable carrier. A preferred fragment is the N-terminal lobe of lactoferrin.

In a fifth aspect, the invention features a method for producing an attenuated vaccine including the steps of (a) contacting an infectious agent with lactoferrin under conditions sufficient to substantially inactivate the infectious agent; and (b) formulating the inactivated infectious agent into a vaccine. Preferably, the lactoferrin is non-pasteurized and/or is isolated from a naturally-occurring source. In a related aspect, the invention features a method for producing an attenuated vaccine including the steps of (a) contacting an infectious agent with a substantially pure fragment of lactoferrin under conditions sufficient to substantially inactivate the infectious agent; and (b) formulating the inactivated infectious agent into a vaccine. A preferred fragment is the N-terminal lobe of lactoferrin.

In a sixth aspect, the invention features an attenuated vaccine including a substantially inactivated infectious agent, where the infectious agent is inactivated with lactoferrin. Preferably, the lactoferrin is non-pasteurized and/or is isolated from a naturally-occurring source. In a related aspect, the invention features an attenuated vaccine including a substantially inactivated infectious agent, where the infectious agent is inactivated with a substantially pure fragment of lactoferrin, such as the N-terminal lobe of lactoferrin.

In a seventh aspect, the invention features a substantially pure peptide consisting of the N-terminal lobe of lactoferrin, where the lobe is isolated from non-pasteurized, naturally-occurring lactoferrin.

By “non-pasteurized” is meant not subjected to the conditions, such as physical (e.g., heat) or chemical (e.g., acid) conditions, that result in sterilization of a milk product.

By “fragment of lactoferrin” is meant an amino acid sequence having antimicrobial activity, but which is shorter than the full-length lactoferrin protein for any given mammalian species.

By “substantially inactivated” is meant that the infectivity or pathogenicity of an agent is reduced, as measured by any standard assay.

By “substantially modified” is meant that all or a portion of one or more of the factors necessary for infectivity are removed from an infectious agent, or degraded.

By “substantially pure” is meant that a compound, such as lactoferrin or a fragment of lactoferrin, has been separated from components which naturally accompany it, or which are generated during its preparation or extraction. For example, a “substantially pure fragment of lactoferrin” is separated from other fragments of lactoferrin. Preferably the lactoferrin preparation is at least 50%, more preferably at least 80%, and most preferably at least 95%, by weight, free from the other proteins, lipids, and other naturally-occurring molecules with which it is naturally associated. The purity of lactoferrin or fragments of lactoferrin can be measured by any appropriate method, for example, column chromatography, polyacrylamide gel electrophoresis, or HPLC analysis.

By “inactivating conditions” are meant the conditions, such as time of treatment, temperature, pH, salt composition, and concentration of lactoferrin or lactoferrin fragment, under which infectious agents can be inactivated by the methods and compositions of the invention.

By “infectious agent” is meant an agent, such as a bacterium or a virus, capable of causing disease in animals.

By “antimicrobial” is meant an agent capable of reducing the infectivity or pathogenicity of any microscopic infectious agent, including, without limitation, any bacteria or virus.

By a “surface protein” is meant a protein or protein-like factor found on or near a surface, such as a cell wall or a virus coat, that contributes to the infectivity of an infectious agent.

By “vaccine” is meant an agent effective to confer the necessary degree of immunity against an infectious agent while preferably causing only very low levels of morbidity or mortality in a host organism population.

By “pharmaceutically acceptable carrier” is meant any standard pharmaceutical carrier, buffer, or excipient currently used, including, without limitation, phosphate buffered saline solution, water, oil/water emulsions, water/oil emulsions, wetting agents, and adjuvants.

The methods and compositions of the invention are useful for inhibiting microbial, including viral, infections. They can be used to selectively inactivate surface proteins of microbes, such as those necessary for colonization or infectivity. At the same time, they leave other microbial activities unchanged, thus providing useful compositions for the preparation of attenuated vaccines.

Other features and advantages of the invention will be apparent from the following detailed description, and from the claims.

### BRIEF DESCRIPTION OF THE DRAWINGS

FIGS. 1A-1D are Western blot analyses showing the effect of lactoferrin on the IgA1 protease precursor. FIG. 1A illustrates removal of the native IgA1 protease precursor and the remnant helper domain from wild-type Rd H. influenzae cells by milk whey; FIGS. 1B and 1C show removal of the IgA1 protease preprotein from H. influenzae Rd3-13 cells by human milk whey; and FIG. 1D demonstrates removal of the IgA1 protease preprotein from H. influenzae cells by recombinant human lactoferrin.

FIGS. 2A-2C are Western blot analyses showing that treatment of H. influenzae strain DB117 with human milk lactoferrin or A. awamori recombinant human lactoferrin results in degradation of the Hap preprotein and Hap. FIG. 2A shows whole cell lysates of H. influenzae strain DB117 derivatives preincubated with PBS alone, and with PBS and 13 μM human milk whey lactoferrin; FIG. 2B illustrates whole cell lysates of H. influenzae strain DB117 derivatives preincubated with PBS alone, and with PBS and 13 μM A. awamori recombinant human lactoferrin; and FIG. 2C shows culture supernatants of H. influenzae strain DB117 derivatives preincubated with PBS alone, and with PBS and 13 μM A. awamori recombinant human lactoferrin.

FIGS. 3A-3C demonstrate the effect of human lactoferrin on Hap-mediated H. influenzae adherence to human epithelial cells. FIG. 3A is a graphical representation showing adherence to Chang epithelial cells by DB117/vector and DB117/HapS243A after incubation in PBS, human milk whey lactoferrin, or recombinant lactoferrin; FIG. 3B is a light micrograph showing DB117/HapS243A adherence to Chang epithelial cell samples after incubation in PBS; and FIG. 3C is a light micrograph showing DB117/HapS243A adherence to Chang epithelial cell samples after incubation in recombinant lactoferrin.

FIG. 4 is a Western blot analysis illustrating the effect of the serine protease inhibitor PMSF on lactoferrin-associated proteolysis of H. influenzae Hap.

FIGS. 5A-5D are Western blot analyses showing that the outer membrane proteins P2, P5, and P6 are not removed by exposure to human milk whey.

FIGS. 6A-6C is a Western blot analysis showing the effect of lactoferrin treatment on epitope tags inserted near the C-terminus of H. influenzae IgA1 protease.

FIG. 7 is a schematic representation of H. influenzae IgA1 protease, highlighting the epitope tags used to define the C-terminal end of the N-Iga fragment released by treatment of H. influenzae Rd or Rd3-13 with human lactoferrin.

FIG. 8 is a Western blot analysis showing the fragment of H. influenzae Hap released by treatment of H. influenzae DB117/pHapS243A with human lactoferrin.

FIG. 9 is a ribbon representation of the three-dimensional structure of human lactoferrin showing the positions of Ser259, Lys73, and Asp315.

FIG. 10 depicts the human lactoferrin catalytic triad before and after rotation of the Lys73 side chain.

FIGS. 11A-11B are Western blot analyses of samples of H. influenzae Rd3-13 after treatment with either wild type N-lobe lactoferrin or N-lobe lactoferrin mutants.

FIGS. 12A-12B are Western blot analyses of whole cells of H. influenzae DB117/pJS106 or DB117/pHapS243A after treatment with either wild type N-lobe lactoferrin or N-lobe lactoferrin mutants.

FIG. 13 is a Western blot analysis demonstrating the removal of H. influenzae protease precursor by human milk whey lactoferrin.

FIG. 14 is a Western blot analysis demonstrating the difference in proteolytic activity between human milk lactoferrin of the invention and commercially available bovine milk lactoferrin.

FIG. 15 is a Western blot analysis comparing commercially available bovine milk lactoferrin preparations and demonstrating the absence of proteolytic activity in the commercially available bovine milk lactoferrin preparations.

FIG. 16 is a Western blot analysis demonstrating the proteolytic activity of recombinant human lactoferrin N-lobe.

### DESCRIPTION OF THE PREFERRED EMBODIMENTS

The invention is based, in part, on the discovery that non-pasteurized lactoferrin isolated from naturally-occurring sources inactivates infectious agents, such as bacteria, without killing the infectious agents. For example, lactoferrin is believed to attenuate the pathogenicity of bacteria by extracting and/or deactivating bacterial cell wall proteins and similar factors that are necessary for colonization and infection, while leaving bacterial viability relatively unaltered. Fragments of lactoferrin can extract and deactivate these proteins as well. In addition, since viruses also have coat proteins that play important roles in their ability to infect host organisms, lactoferrin and its fragments are useful for inactivating viruses as well.

In one particular example, lactoferrin removes the H. influenzae IgA1 protease preprotein from bacterial cell walls. Once this protein is separated from the cell wall, it can be inhibited by milk anti-IgA1 protease antibodies. Lactoferrin and its fragments also proteolytically degrade, and therefore inactivate, the Hap adhesin on the bacterial surface, thereby diminishing the ability of the bacteria to adhere to epithelial cells.

It is believed that the ability of lactoferrin and its fragments to extract and degrade membrane proteins is dependent, at least in part, on proteolysis; i.e., that lactoferrin is a protease or acquires proteolytic activity through interaction with target proteins. This hypothesis is supported the observation that both extraction and degradation are inhibited by pretreatment of lactoferrin preparations with phenylmethylsulphonyl fluoride (PMSF), a serine protease inhibitor.

Fragments of lactoferrin can also be used to extract and degrade these membrane proteins. There is no minimum size for the fragments that can be used; the only requirement is that the fragments are large enough to retain proteolytic activity. Lactoferrin fragments may be generated by standard techniques of molecular biology (e.g., by standard deletion procedures) or, particularly for short fragments, by chemical synthetic approaches. Once generated, the fragments are tested for proteolytic activity using any standard assay (including the assays described herein). Glycosylation of these fragments may be accomplished in vivo by production in an appropriate host cell (e.g., a mammalian host cell) or in vitro using purified glycosylation enzymes or cell extracts.

In some applications, however, larger fragments of lactoferrin are preferred. For example, fragments of lactoferrin having at least 100, and more preferably 200, amino acid residues are preferred for some uses.

The active sites responsible for the extraction/degradation capabilities appear to reside on the N-terminal lobe of lactoferrin, which has 334 amino acids. Therefore, fragments of lactoferrin including at least a portion of the N-terminal lobe and having this activity are also useful in the invention. A preferred fragment is the N-terminal lobe itself; this lobe has been shown to be as effective as full-length lactoferrin in extracting the H. influenzae IgA1 protease from bacterial cells and in degrading the Hap adhesin.

Lactoferrin from a variety of sources may be used in the methods and compositions described herein; both lactoferrin isolated from natural sources and recombinant lactoferrin have been shown to exhibit proteolytic activity. It is important that the lactoferrin not be pasteurized, as pasteurization destroys the proteolytic activity of lactoferrin. Exemplary sources of lactoferrin include bovine milk and human milk. Lactoferrin and fragments of lactoferrin can also be produced using synthetic or recombinant methods, for example, as described in Stowell et al., Biochem. J. 276: 349-355 (1991). When recombinant lactoferrin or lactoferrin fragments are used, it is important that they are glycosylated in a manner similar to that of naturally-occurring lactoferrin, for example, using the methods described above.

Targets for lactoferrin include any number of surface proteins. Exemplary proteins include IgA1 protease and Hap, which belong to the same family of gram-negative bacterial autotransported secretory proteins. IgA proteases are the prototypes of a family of gram-negative bacterial proteins that undergo autosecretion, as described in Jose et al., Mol. Microbiol. 18: 378-380 (1995). The H. influenzae strain Rd protease described herein is synthesized as a 185 kDa protein with four domains, including an N-terminal signal sequence, a central serine protease (IgAp), a highly basic and helical alpha domain (IgAα), and a carboxy-terminal beta or helper domain (IgAβ) (Poulsen et al., Infect. Immun. 57: 3097-3105 (1989); Pohlner et al., Nature 325: 458-462 (1987)). The signal sequence directs export across the bacterial inner membrane, and is then cleaved. Subsequently, the remainder of the protein (hereafter called the preprotein) inserts into the outer membrane via the beta domain. This domain is predicted to form a β-barrel structure with a hydrophilic channel, thus facilitating translocation of the protease and the alpha domain to the extracellular space. Ultimately, the protease domain gains catalytic activity and cleaves within the alpha domain to release itself from the surface of the organism.

The H. influenzae Hap protein is a nonpilus protein that promotes intimate interaction with human epithelial cells (St. Geme III et al., Mol. Microbiol. 14: 217-233 (1994)). It was originally identified by its ability to confer the capacity for in vitro attachment and invasion when expressed in a nonadherent, noninvasive laboratory strain of H. influenzae. Hap shares significant sequence homology (30-35% identity and 51-55% similarity) with the H. influenzae and Neisseria gonorrhoeae IgA1 proteases, and undergoes autosecretion via an analogous pathway. It is produced as a 155 kDa protein with three functional domains, including an N-terminal signal sequence, a surface localized serine protease domain (Hap_{S}), and a C-terminal outer membrane domain (Hapβ) (Hendrixson et al., Mol. Microbiol. 26: 505-518 (1997)). Ultimately, the Hap_{S} domain mediates an autoproteolytic cleavage event, releasing itself from Hap and from the surface of the organism. It is believed that attachment to host epithelial cells is a function of the preprotein (Hap_{S} linked to Hapβ), prior to autoproteolytic cleavage. Lactoferrin inactivates this protein by degrading the Hap_{S} domain.

Thus, these proteins both contain a C-terminal domain that is embedded in the membrane, and an N-terminal protease domain that resides on the surface of the organism until released to the extracellular medium by autoproteolysis of the preprotein (the passenger domain). Proteins sharing these characteristics are also expected to be affected by lactoferrin or its fragments. Examples of such proteins include the polyprotein precursors of Neisseria gonorrheae, H. mustelae, Bordetella spp., Serratia marcescens, Helicobacter pylori, E. coli, S. flexneri, and B. pertussis.

Because certain other outer membrane proteins of H. influenzae are resistant to the proteolytic effects of lactoferrin, the presence of the N-terminal passenger domain may be important for interaction with lactoferrin and its fragments. For example, P2, P5, and P6 are H. influenzae outer membrane proteins that, like IgAβ and Hapβ, are believed to form β-barrel structures. P2, P5, and P6, however, lack N-terminal passenger domains. These proteins are unaffected by lactoferrin.

Lactoferrin recognizes arginine-rich sequences in the H. influenzae IgA1 protease and Hap proteins. N-terminal amino acid sequencing revealed the sequence ALVRDDV, corresponding to the predicted amino terminus of the IgA1 protease precursor protein (after cleavage of the signal sequence). Based on this information, the released form of IgA1 protease was designated N-Iga. To extend this result, derivatives of strain Rd or strain Rd3-13 were generated, containing a 6[His] or RGS-6[His] eptitope tag in IgA1 protease in place of amino acids 1044-1049, 1251-1260, 1540-1550, or 1628-1638. These organisms were then treated with milk lactoferrin, and the whole cells and culture supernatants were examined by Western analysis with either antiserum #331 (against IgA1 protease) or anti-RGS.6[His].

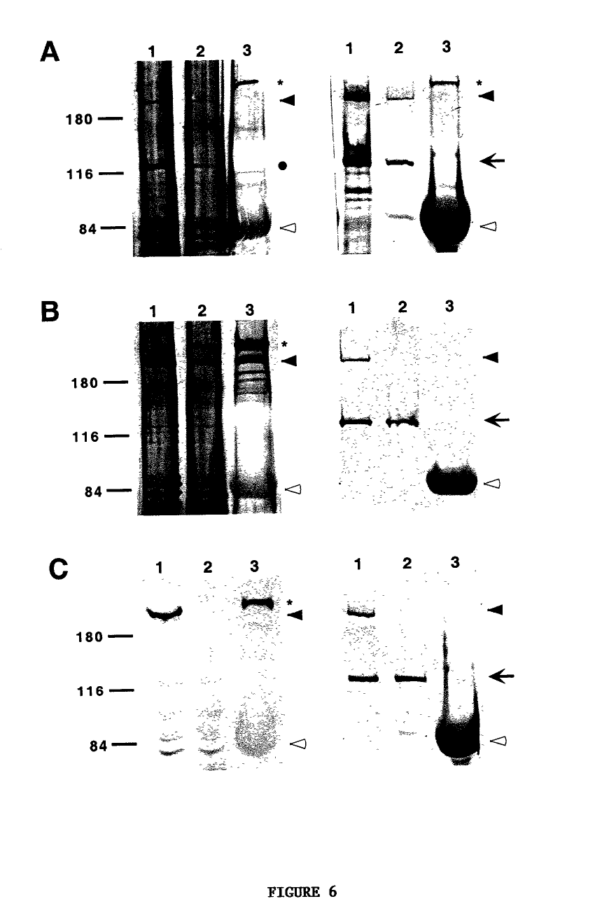

FIG. 6A shows Western analysis of strain Rd expressing IgA1 protease with epitope D. FIG. 6B shows Western analysis of strain Rd3-13 expressing IgA1 protease with epitope E. FIG. 6C shows Western analysis of strain Rd3-13 expressing IgA1 protease with epitope F. In each panel, the first set of 3 lanes was probed with rabbit polyclonal antiserum #331, which reacts with the IgA1 protease precursor protein, Igaₚ (the secreted passenger domain), and Iga_{β} (the C-terminal domain), and the second set of 3 lanes was probed with anti-RGS.6[His]. In each panel, samples were loaded as follows: lane 1, whole cells, no treatment; lane 2, whole cells, after treatment with lactoferrin; lane 3, culture supernatant, after treatment with lactoferrin. The arrowhead indicates the IgA1 protease precursor protein, the asterisk indicates the N-Iga fragment generated by treatment with lactoferrin, the blackened circle indicates Iga_{β}, the arrow indicates an Rd cellular protein that reacts with anti-RGS.6[His], and the open arrowhead indicates lactoferrin.

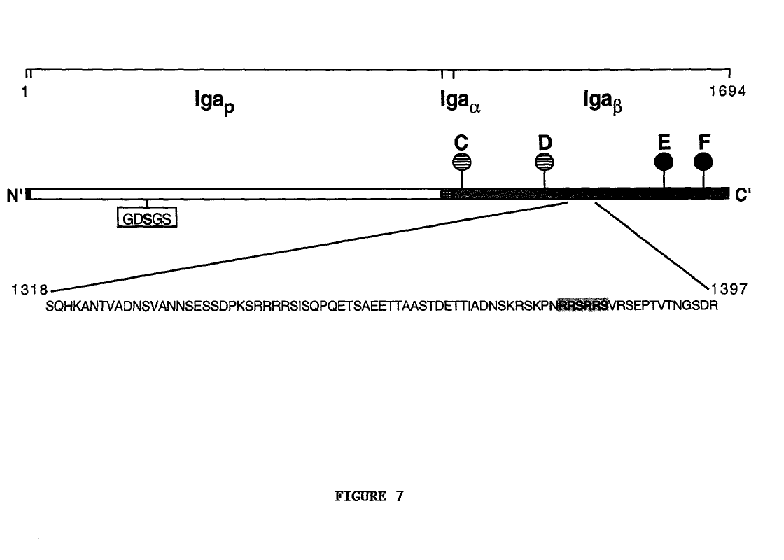

In FIG. 7, H. influenzae IgA1 protease is divided into several structural regions, including the signal sequence (black bar), the Igaₚ domain (white bar), the Iga_{α} domain (checkered bar), and the Iga_{β} domain (gray bar). The active site serine at residue 288 in the Igaₚ domain is indicated in bold. The locations of 6[His] and RGS-6[His] eptitope tags are shown with lollipops. Striped lollipops represent tags that are present in N-Iga, and black lollipops indicate tags that are removed from IgA1 protease by treatment with lactoferrin and are absent in N-Iga. Specific locations are as follows: C, amino acids 1044-1049; D, amino acids 1251-1260; E, amino acids 1540-1550; and F, amino acids 1628-1638. The amino acid sequence in the lower portion of the figure corresponds to the peptides that were synthesized to define specific cleavage sites, with the arginine-rich region in bold. As shown in FIGS. 6 and 7, both antiserum 331 and anti-RGS-6[His] detected N-Iga with the epitope tag at amino acids 1044-1049 (position C) or 1251-1260 (position D). In contrast, only antiserum #331 detected N-Iga with the epitope tag at amino acids 1540-1550 (position E) or 1628-1638 (position F). Considered together, these results suggest that N-Iga is an N-terminal fragment of the IgA1 protease precursor protein, arising by lactoferrin cleavage of the protein somewhere between residues 1260 and 1540 (after the epitope tags at positions C and D, before the epitope tags at positions E and F).

To further define the site of cleavage of the IgA1 protease precursor protein, N-Iga was resolved on an SDS-PAGE gel, then stained with Coomassie blue and excised. The excised band was subjected to Lys-C digestion, and mass spectrometry was performed. As shown in Table 1, this approach identified predicted fragments of the IgA1 protease precursor protein from the N-terminus up to and including the fragment containing amino acids Ser1341-Lys1373, suggesting that cleavage by lactoferrin occurs somewhere after Lys1373. To extend this result, a series of peptides fragments of IgA1 protease were synthesized, in all cases including the residue Arg1397 at the very C terminus and extending either 20, 40, 60 or 80 residues towards the N terminus (see FIG. 7). Subsequently, these peptides were treated with lactoferrin and then examined the reaction mixtures by mass spectrometry. Lactoferrin treatment of the 60 amino acid peptide gave rise to two abundant large fragments, consistent with a 46-mer and a 47-mer, beginning with N-terminal Asp1338 and ending with C-terminal RRSR and RSRR, respectively. This finding indicated cleavage between Arg1382 and Arg1383 and between Arg1383 and Ser1384. Similar to results with the 60 amino acid peptide, lactoferrin treatment of the 80, 40, and 20 amino acid peptides gave rise to fragments indicating cleavage between Arg1382 and Arg1383 and between Arg1383 and Ser1384.

TABLE 1
Assignment of proteolytic fragments of Lys-C-digested
N-Iga after mass spectrometry

| Fragment mass | Assigned peptide | Calculated mass |
|---|---|---|
| 1035.82 | Unassigned | N/A |
| 1041.21 | Unassigned | N/A |
| 1134.56 | Unassigned | N/A |
| 1196.36 | Unassigned | N/A |
| 1297.51 | Unassigned | N/A |
| 1352.93 | 925-937 | 1352.492 |
| 1468.97 | 1225-1238 | 1468.612 |
| 1538.30 | Unassigned | N/A |
| 1600.65 | 250-264 | 1600.737 |
| 1629.97 | 624-637 | 1629.751 |
| 1635.86 | 45-59 | 1635.827 |
| 1642.88 | 161-172 | 1642.884 |
| 1658.69 | Unassigned | N/A |
| 1672.21 | 85-99 | 1672.031 |
| 1690.09 | 234-249 | 1689.912 |
| 1791.64 | 1202-1216 | 1791.832 |
| 1813.74 | Unassigned | N/A |
| 1827.08 | 318-331 | 1827.056 |
| 1833.97 | 198-213 | 1834.135 |
| 1990.33 | Unassigned | N/A |
| 2070.10 | 124-140 | 2070.190 |
| 2080.41 | 977-993 | 2080.316 |
| 2093.11 | 493-512 | 2093.353 |
| 2113.76 | 337-356 | 2113.212 |
| 2200.51 | Unassigned | N/A |
| 2231.38 | 1281-1302 | 2231.386 |
| 2294.82 | 595-613 | 2295.565 |
| 2315.95 | Unassigned | N/A |
| 2383.38 | 703-725 | 2383.723 |
| 2398.45 | Unassigned | N/A |
| 2423.74 | Unassigned | N/A |
| 2511.31 | Unassigned | N/A |
| 2526.76 | Unassigned | N/A |
| 2547.75 | 1059-1082 | 2547.657 |
| 2597.88 | 1083-1106 | 2597.671 |
| 2617.27 | 173-197 | 2616.763 |
| 2646.90 | 357-381 | 2646.744 |
| 2719.84 | 727-748 | 2719.896 |
| 2775.70 | Unassigned | N/A |
| 2847.39 | Unassigned | N/A |
| 2868.43 | Unassigned | N/A |
| 2958.58 | 644-668 | 2958.232 |
| 2975.13 | Unassigned | N/A |
| 2991.51 | Unassigned | N/A |
| 3021.58 | Unassigned | N/A |
| 3323.80 | Unassigned | N/A |
| 3625.71 | 1341-1373 | 3625.815 |
| 3663.98 | Unassigned | N/A |
| 3999.62 | 1152-1189 | 4000.247 |
| 4241.95 | 557-594 | 4241.671 |
| 4544.10 | 1239-1280 | 4543.870 |
| 4761.03 | Unassigned | N/A |
| 5734.59 | Unassigned | N/A |
| 5756.03 | Unassigned | N/A |
| 6042.02 | 994-1050 | 6042.534 |
| 6063.27 | Unassigned | N/A |

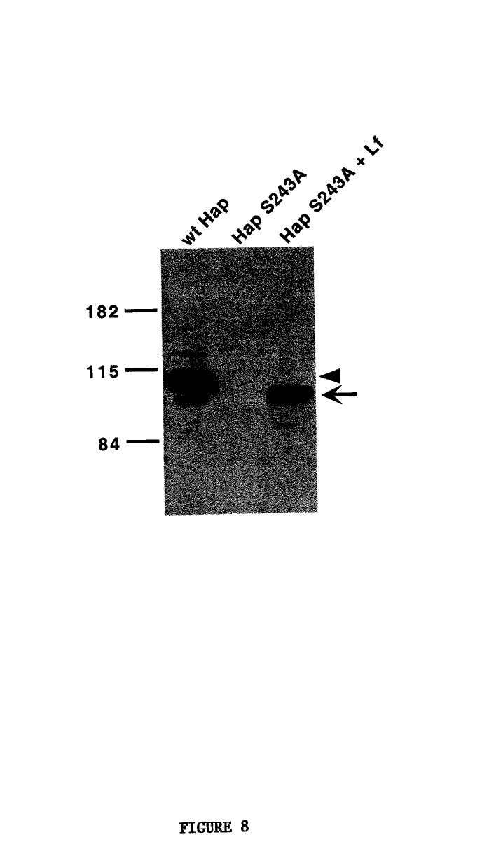

In light of the results with IgA1 protease, the Hap amino acid sequence was examined for regions homologous to RRSRRSVR. A similar arginine-rich region is present between amino acids Val1016 and Arg1023, with the specific sequence including VRSRRAAR. This arginine-rich region is very close to the Leu1036-Gln1037 peptide bond of Hap, which is the primary site at which cleavage occurs during autoproteolysis of the precursor protein (Hendrixson et al., 1997). Of note, lactoferrin cleavage of Hap results in release of a fragment that migrates at slightly less than 110 kDa, very close in size to the Hap_{S} domain that is released during natural autoproteolysis (FIG. 8). FIG. 8 depicts a Western blot analysis showing the fragment of H. influenzae Hap released by treatment of H. influenzae DB117/pHapS243A with human lactoferrin. Culture supernatants were precipitated using trichloroacetic acid and were examined by Western analysis using antiserum GP74, which was raised against purified Hap_{S}. Lane 1 shows Hapₛ from strain DB117/pJS106 (wild type hap) following autoproteolysis and serves as a control. Lane 2 shows DB117/pHapS243A after incubation in PBS. Lane 3 shows DB117/pHapS243A after treatment with human lactoferrin. The arrowhead indicates Hap_{S}, and the arrow indicates the fragment of Hap released by treatment with lactoferrin.

In an effort to define the site within Hap at which lactoferrin cleaves, DB117/pHapS243A was treated with either milk lactoferrin or N-lobe lactoferrin, and the fragment released into the culture supernatant was recovered. This fragment was then resolved by SDS-PAGE and stained with Coomassie blue. Subsequently, the fragment was excised and subjected to trypsin digestion. Ultimately, the tryptic digest was examined by mass spectrometry, which revealed the predicted fragments of the Hap precursor protein from the N-terminus up to and including the fragment containing amino acids 991-1008, suggesting cleavage somewhere after amino acid 1008 (Table 2).

TABLE 2
Assignment of proteolytic fragments of trypsin-digested
Hap after mass spectrometry

| Fragment mass | Assigned peptide | Calculated mass |
|---|---|---|
| 1158.99 | 940-949 | 1158.296 |
| 1182.29 | 349-358 | 1182.277 |
| 1206.17 | Unassigned | N/A |
| 1211.63 | Unassigned | N/A |
| 1222.22 | 71-81 | 1222.444 |
| 1226.49 | 651-660 | 1226.417 |
| 1238.36 | Unassigned | N/A |
| 1260.57 | 228-240 | 1260.432 |
| 1264.30 | 469-480 | 1264.423 |
| 1297.51 | Unassigned | N/A |
| 1334.42 | 281-290 | 1334.469 |
| 1369.62 | 964-974 | 1369.563 |
| 1387.62 | Unassigned | N/A |
| 1401.82 | 505-516 | 1401.608 |
| 1463.59 | 580-591 | 1463.673 |
| 1515.60 | 502-515 | 1515.712 |
| 1552.60 | 908-921 | 1552.681 |
| 1558.78 | 950-963 | 1158.690 |
| 1574.04 | 359-371 | 1573.760 |
| 1590.32 | Unassigned | N/A |
| 1672.31 | 502-516 | 1671.899 |
| 1683.77 | Unassigned | N/A |
| 1696.58 | Unassigned | N/A |
| 1773.25 | Unassigned | N/A |
| 1785.89 | 344-358 | 1785.959 |
| 1822.29 | 265-280 | 1821.987 |
| 1861.00 | 592-609 | 1861.145 |
| 1875.45 | 726-743 | 1875.045 |
| 1913.75 | 131-145 | 1914.000 |
| 1940.36 | 651-668 | 1940.209 |
| 1950.39 | 265-281 | 1950.161 |
| 1955.88 | 975-990 | 1956.211 |
| 1967.90 | Unassigned | N/A |
| 2000.12 | 991-1008 | 2000.216 |
| 2000.31 | 612-628 | 2000.269 |
| 2017.12 | 922-939 | 2017.240 |
| 2083.21 | 435-455 | 2083.308 |
| 2156.31 | 612-629 | 2156.457 |
| 2164.08 | Unassigned | N/A |
| 2185.43 | 610-628 | 2185.495 |
| 2238.02 | 561-579 | 2237.395 |
| 2293.09 | 282-299 | 2293.497 |
| 2305.29 | Unassigned | N/A |
| 2321.23 | Unassigned | N/A |
| 2368.86 | Unassigned | N/A |
| 2387.87 | Unassigned | N/A |
| 2421.67 | 281-299 | 2421.671 |
| 2433.43 | 127-145 | 2433.558 |
| 2440.81 | 886-907 | 2440.758 |
| 2456.10 | Unassigned | N/A |
| 2476.47 | Unassigned | N/A |
| 2534.37 | 863-885 | 2534.742 |
| 2542.53 | 481-501 | 2542.766 |
| 2573.48 | Unassigned | N/A |
| 2598.32 | 630-650 | 2598.851 |
| 2614.60 | 47-70 | 2614.034 |
| 2628.19 | Unassigned | N/A |
| 2698.01 | 940-963 | 2697.964 |
| 2703.98 | 456-480 | 2703.968 |
| 2723.95 | Unassigned | N/A |
| 2740.44 | 241-264 | 2740.149 |
| 2774.70 | 188-212 | 2774.882 |
| 2812.43 | Unassigned | N/A |
| 2829.00 | 748-773 | 2829.101 |
| 2868.24 | Unassigned | N/A |
| 3030.50 | Unassigned | N/A |
| 3048.90 | 774-802 | 3048.249 |
| 3117.40 | Unassigned | N/A |
| 3132.85 | Unassigned | N/A |
| 3156.38 | Unassigned | N/A |
| 3198.57 | 744-773 | 3198.565 |
| 3215.83 | Unassigned | N/A |
| 3305.56 | 580-609 | 3305.796 |
| 3375.44 | Unassigned | N/A |
| 3392.33 | 184-212 | 3392.590 |
| 3471.47 | Unassigned | N/A |
| 3532.23 | Unassigned | N/A |
| 3932.54 | Unassigned | N/A |
| 3972.35 | 82-118 | 3972.235 |
| 4038.70 | Unassigned | N/A |
| 4195.35 | Unassigned | N/A |
| 4511.17 | Unassigned | N/A |
| 4673.42 | Unassigned | N/A |
| 4695.36 | Unassigned | N/A |
| 4798.18 | 517-560 | 4798.298 |

Next, a 20 amino acid peptide corresponding to residues Ala1007-Phe1026 of Hap was synthesized and subjected to cleavage by milk lactoferrin. As predicted from the homology with IgA1 protease, analysis of the reaction mixture by mass spectrometry revealed the 13-mer corresponding to N′-AKTQTGEPKVRSR and the 14-mer corresponding to N′-AKTQTGEPKVRSRR, indicating cleavage between Arg1019 and Arg1020 and between Arg1020 and Ala1021.

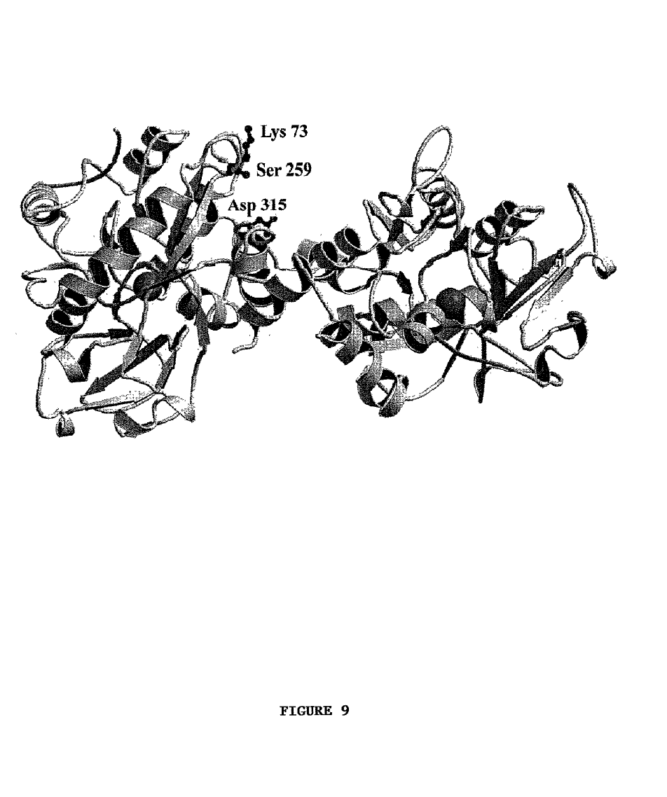

The N-lobe of lactoferrin contains a surface serine with neighboring lysine and aspartic acid residues. In considering the mechanism of human lactoferrin proteolytic activity, it is noteworthy that the primary amino acid sequence of lactoferrin lacks signature features of known proteases. Nevertheless, previous studies found that preincubation with either diisopropyl fluorophosphate (DFP) or phenylmethylsulfonyl fluoride (PMSF) resulted in inhibition of proteolytic activity, suggesting the possibility that lactoferrin is a serine protease (Qiu et al., 1998). To explore this hypothesis, the human lactoferrin crystal structure was examined for surface serine residues. As shown in FIG. 9, the left-hand portion of the molecule is the N-lobe, and the right-hand portion of the molecule is the C-lobe, with a cleft in between. Ser259 is present on the surface of the N-lobe and is adjacent to Lys73 and Asp315, projecting into the large cleft that exists between the N-lobe and the C-lobe of the native structure. Although serine proteases typically contain a catalytic triad consisting of serine, histidine, and aspartic acid, it is possible that lysine is capable of substituting for histidine as a general base in the proteolytic reaction. Alternatively, Ser259 and Lys73 might form a catalytic dyad, as has been described for the mitochondrial and prokaryotic signal peptidase I enzymes (Paetzel, M. & Strynadka, N. C. J. (1999) Common protein architecture and binding sites in proteases utilizing a Ser/Lys dyad mechanism. Protein Sci. 8, 2533-2536).

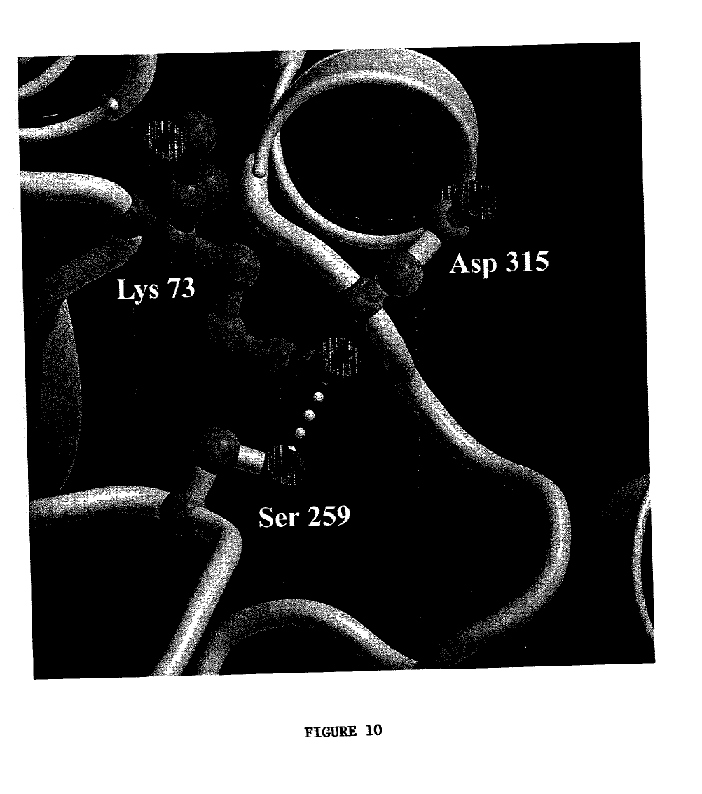

Further examination of the lactoferrin crystal structure revealed that the ε-amino group of Lys73 is relatively remote from the O_{γ} of Ser259 (7.5-9 Å away). However, the Lys73 side chain is very mobile (B factors 60-80 Å²), and simple rotation using standard rotamers could place the ε-amino group in the space between Ser259 and Asp315, close enough for hydrogen bonding with Ser259 O_{γ}. The result of this rotation would be a serine-lysine-aspartic acid triad, with all three side chains exposed to the solvent and in position to mediate proteolytic cleavage (FIG. 10). FIG. 10 shows the mobility of the Lys73 side chain, and simple rotation using standard rotamers places the ε-amino group in the space between Ser259 and Asp315, close enough for hydrogen bonding with Ser259 O_{γ}. The position of the Lys73 side chain in the crystal structure is shown directly above the designation “Lys 73”, and the position of this side chain after putative rotation is shown participating in hydrogen bonding (indicated by a dotted line) with the oxygen atom in Ser259.

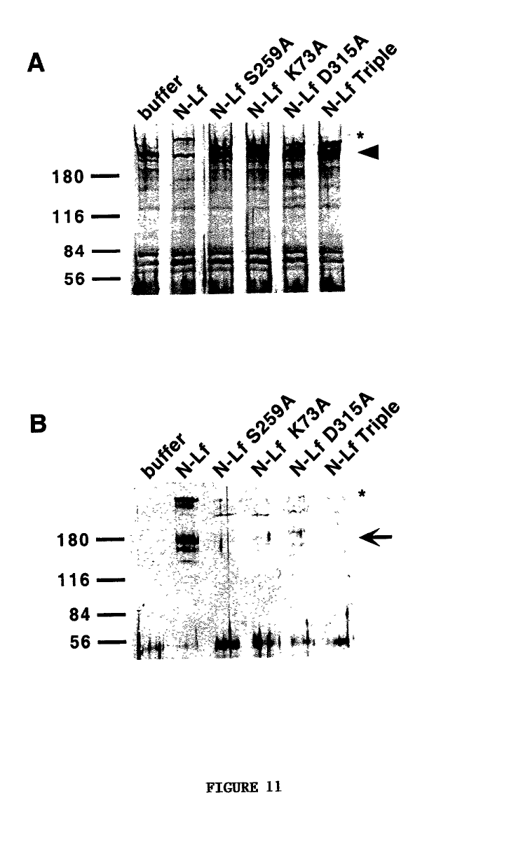

Mutagenesis of lactoferrin Ser259, Lys73, and Asp315 affects proteolysis of the H. influenzae IgA1 protease and Hap proteins. To explore the roles of Ser259, Lys73, and Asp315 in lactoferrin proteolytic activity, site-directed mutagenesis was performed, generating four mutant N-lobe proteins. Each of these residues was changed individually, and then in combination. Following purification from BHK cell culture supernatants, the mutant proteins were examined for the ability to cleave H. influenzae IgA1 protease. As a control, an unrelated mutant N-lobe protein with a mutation at Pro251, a residue remote from the putative active site, was examined. As shown in FIG. 11, incubation of whole bacteria with wild type N-lobe lactoferrin resulted in efficient removal of IgA1 protease from the bacterial membrane. In contrast, mutation of Ser259, Lys73, and Asp315 either individually or together eliminated the ability of lactoferrin to cleave the IgA1 protease precursor protein (FIG. 11). FIG. 11A shows the analysis of whole cells, and FIG. 11B shows culture supernatants. Western analysis was performed with rabbit polyclonal antiserum #331, which reacts with the IgA1 protease precursor protein and Igaₚ (the secreted passenger domain). The arrowhead indicates the IgA1 protease precursor protein, the asterisk indicates the N-Iga fragment generated by treatment with lactoferrin, and the arrow indicates a breakdown product of N-Iga. Mutation of Pro251 had no effect on lactoferrin proteolysis of IgA1 protease.

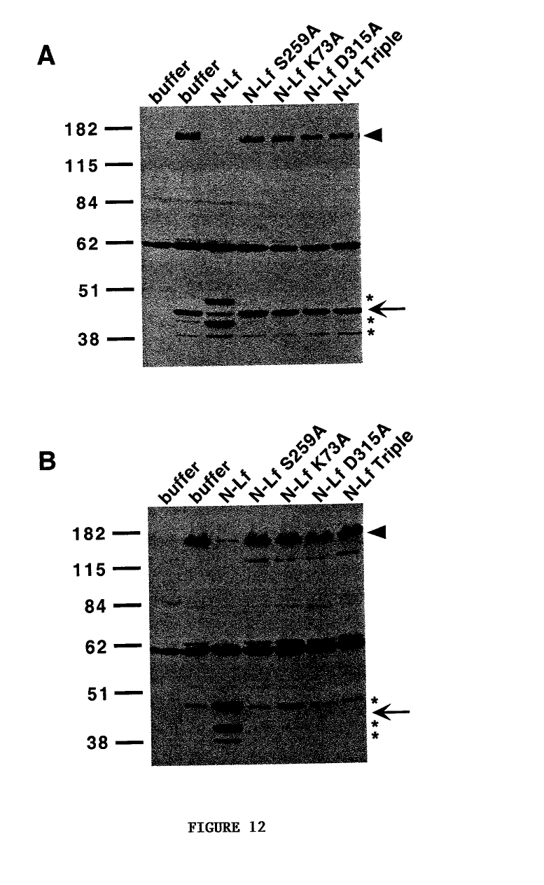

To extend these results, the mutant proteins were examined in assays with bacteria expressing either wild type Hap or HapS243A. FIG. 12A shows whole cells of strain H. influenzae DB117/pJS106 (expressing wild type Hap), and FIG. 12B shows whole cells of strain H. influenzae DB117/pHapS243A (expressing HapS243A). In both panels, the first lane contains samples from DB117/pGJB103 (vector). Western analysis was performed with rabbit polyclonal antiserum Rab730, which reacts with the Hap precursor protein, Hapₛ, and Hap_{β}. The arrowhead indicates the Hap precursor protein, the arrow indicates Hap_{β}, and the asterisks indicate C-terminal fragments of Hap generated by treatment with lactoferrin. As shown in FIG. 12, wild type lactoferrin N-lobe cleaved full-length Hap and generated cell-associated fragments of 47 kDa, 43 kDa, and 39 kDa. On the other hand, with N-Lf(S259A), N-Lf(D315A), and N-Lf(triple mutant), full-length Hap remained virtually unaffected. With N-Lf(K73A), cleavage of full-length Hap was detectable but remained minimal. Consistent with these observations, in experiments with DB117/pHapS243A, preincubation with wild type N-lobe lactoferrin resulted in an 80% decrease in adherence of bacteria to A4549 respiratory epithelial cells, while preincubation with either N-Lf(S259A), N-Lf(D315A), or N-Lf(triple mutant) had no appreciable effect on Hap-mediated adherence (Table 3). Preincubation with N-Lf(K73A) resulted in a small decrease in adherence, consistent with the residual activity that this mutant has against the Hap substrate (Table 3).

TABLE 3
Effect of mutant N-lobe lactoferrin on Hap-mediated
adherence to A549 epithelial cells.

| Strain | Treatment | Adherence (% inoculum)* |
|---|---|---|
| DB117/pGJB103 | PBS | 4.1 ± 0.9 |
| DB117/pHapS243A | PBS | 58.1 ± 4.1 |
| DB117/pHapS243A | Wild type N-Lf† | 7.1 ± 2.1 |
| DB117/pHapS243A | N-Lf(S259A) | 70.2 ± 2.5 |
| DB117/pHap5243A | N-Lf(K73A) | 45.0 ± 3.8 |
| DB117/pHapS243A | N-Lf(D316A) | 59.1 ± 4.1 |
| DB117/pHapS243A | N-Lf(triple mutant) | 61.4 ± 6.8 |

*Adherence was determined in a 30 minute assay as described previously (St. Geme et al., 1993). Values represent means ± standard errors of the means of three measurements from a representative experiment.
†N-Lf refers to the N-lobe of lactoferrin.

These results demonstrate that mutation of Lys73 to alanine resulted in reduced lactoferrin cleavage of Hap but failed to abolish proteolysis altogether. In most serine proteases with a catalytic triad, mutation of the serine or the histidine results in a 10⁶-fold decrease in activity, while mutation of the aspartic acid is associated with a 10⁴-fold decrease (Carter, P. & Wells, J. (1988) Dissecting the catalytic triad of a serine protease. Nature 332, 564-568; Corey, D. R. & Craik, C. S. (1992) An investigation into the minimum requirements for peptide hydrolysis by mutation of the catalytic triad of trypsin. J. Am. Chem. Soc. 114, 1784-1790; Craik et al., (1987) The catalytic role of the active site aspartic acid in serine proteases. Science 237, 909-913). For the most part, in serine proteases with a catalytic dyad, mutation of either the serine or the lysine is associated with a less dramatic effect on activity, reflecting the fact that these proteases are known to be are relatively inefficient in the first place (Paetzel, M. & Dalbey, R. E. (1997) Catalytic hydroxyl/amine dyads with serine proteases. Trends Biochem. Sci. 22, 28-31). Nevertheless, one possibility is that Lys73 is dispensable for cleavage of Hap, with Hap itself contributing a histidine or lysine residue itself to the catalytic mechanism. Indeed, so-called substrate-assisted catalysis has been described with subtilisin. In particular, a subtilisin mutant lacking histidine can cleave substrates containing histidine because the substrate histidine participates directly in the catalytic process.

Based on these mutagenesis studies, lactoferrin is distinct from existing known serine proteases and appears to be a hybrid protein, with a catalytic triad that contains a lysine in place of histidine. The cleft that neighbors Ser259, Lys73, and Asp315 and lies between the N-lobe and the C-lobe of human lactoferrin is 12-15 Å across, sizeable enough to accommodate a large polypeptide substrate. The catalytic region is completely removed from either iron binding site. In addition to Asp315, several other acidic residues are within 10 Å of Ser259, including Glu264 and Asp265 in the N-lobe and Asp379 and Glu388 in the C-lobe. It is possible that these acidic residues contribute to account for the preferred cleavage of arginine-rich regions.

To utilize a lysine as a general base, an enzyme must provide an environment such that the pKₐ of that lysine is depressed, allowing the lysine to be maintained in the unprotonated state. A nearby positive charge from another lysine or an arginine represents one means by which depression of the pKₐ is can be accomplished mechanism by which the pKₐ is depressed (Paetzel and Strynadka, 1999). Burial of the ε-amino group of lysine within the enzyme/substrate complex is another mechanism, as illustrated by the type 1 signal peptidases (Paetzel and Strynadka, 1999). In the case of lactoferrin, it is possible that Arg75, Arg258, and Arg313 provide positive charges that influence the pKₐ of Lys73. Alternatively, the density of arginine residues at the cleavage site in IgA1 protease and Hap and other potential substrates may lower the pKₐ sufficiently, thus explaining the preference for cleavage at arginine-rich sequences.

In this study we found that lactoferrin cleaved both IgA1 protease and Hap after the sequence —RSRR— or —RRSR—. Cleavage of substrates with positively charged residues at in the P1 and P4 (the first and fourth positions N-terminal to the scissile bond) positions is reminiscent of a family of calcium-dependent endoproteases referred to as the subtilisin-like proprotein convertases (Gensberg et al. (1998) Subtilisin-related serine proteases in the mammalian constitutive secretory pathway. Sem. Cell Devel. Biol. 9, 11-17; Krysan et al. Krysan, D. J., Rockwell, N. C., & Fuller, R. S. (1999) Quantitative characterization of furin specificity. J. Biol. Chem. 274, 23229-23234). The original member of this family is a yeast protein called Kex2, and the prototype in humans is an intracellular enzyme referred to as furin (Julius et al. (1984) Cell 37, 1075-1089; van den Ouweland et al. (1990) Structural homology between the human fur gene product and the subtilisin-like protease encoded by yeast KEX2. Nucleic Acids Res. 18, 664). These proteins mediate limited proteolytic cleavage of growth factors, prohormones, proneuropeptides, zymogens, and adhesion molecules, to name a few examples, and influence a variety of fundamental cellular functions. Recent evidence indicates that furin-like cellular enzymes may also be involved in activation of viral envelope proteins (Steineke-Grober et al. (1992) Influenza virus hemagglutinin with multibasic cleavage site is activated by furin, a subtilisin-like endoprotease. EMBO J. 11, 2407-2714). By analogy to the proprotein convertases, it is possible that lactoferrin cleaves substrates inside the cell as well.

In this work, we established that lactoferrin cleaves IgA1 protease within the arginine-rich region between residues 1379 and 1386 and Hap in the arginine-rich region between residues 1016 and 1023. As highlighted in FIG. 12, it is likely that lactoferrin recognizes other sites in Hap and possibly IgA1 protease as well, as cleavage results in 3 different C-terminal fragments of Hap. Along these lines, it is interesting to note additional arginine-rich sequences at amino acids 857-862 and 1058-1064 in Hap are potential sites for cleavage, perhaps accounting in part for the other C-terminal fragments observed.

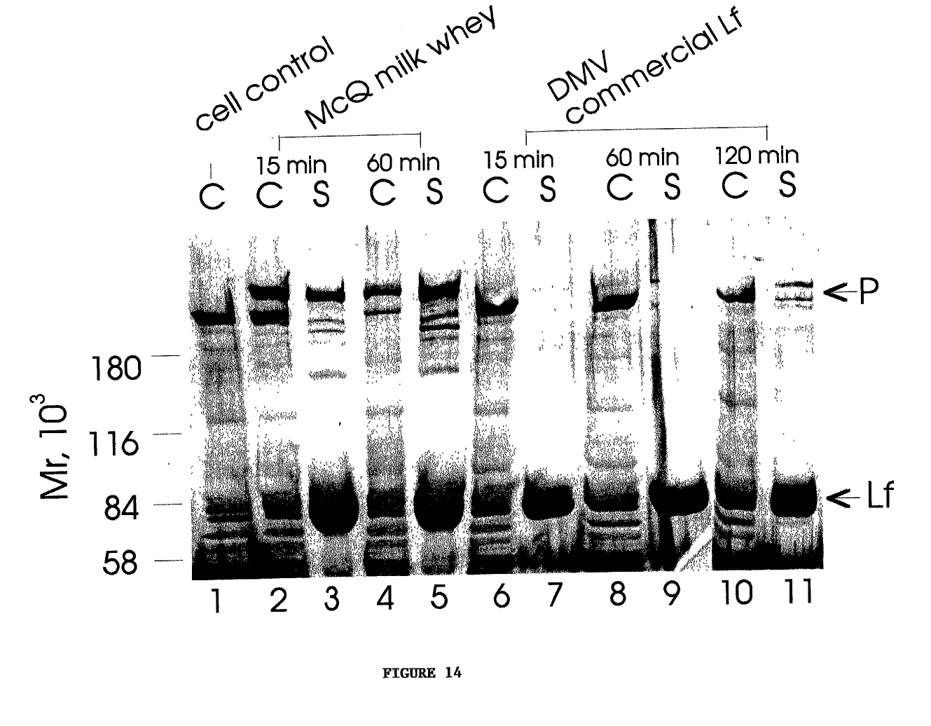

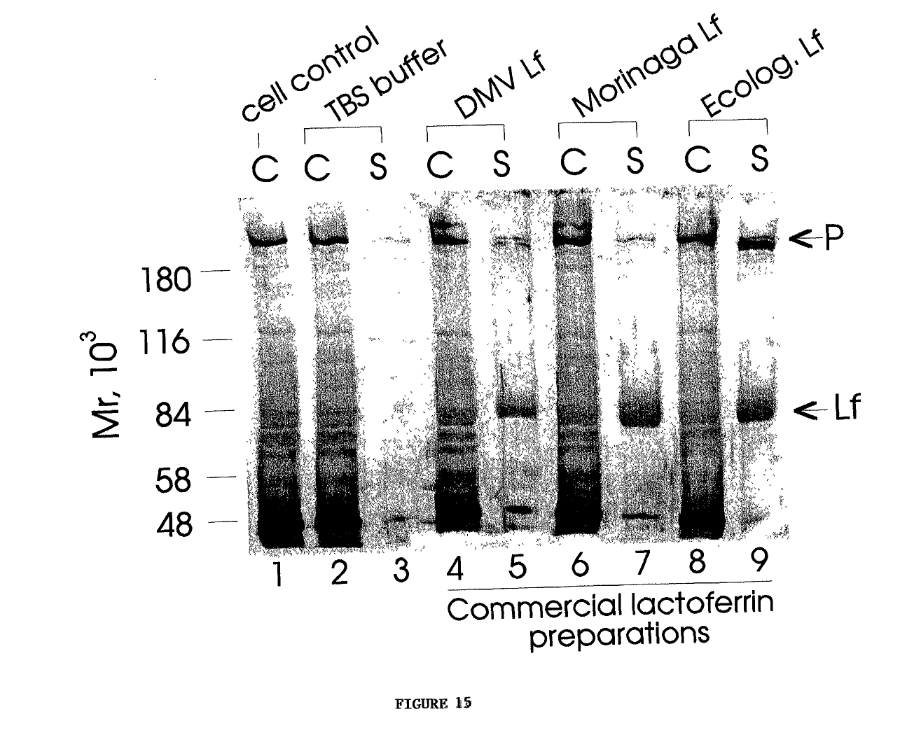

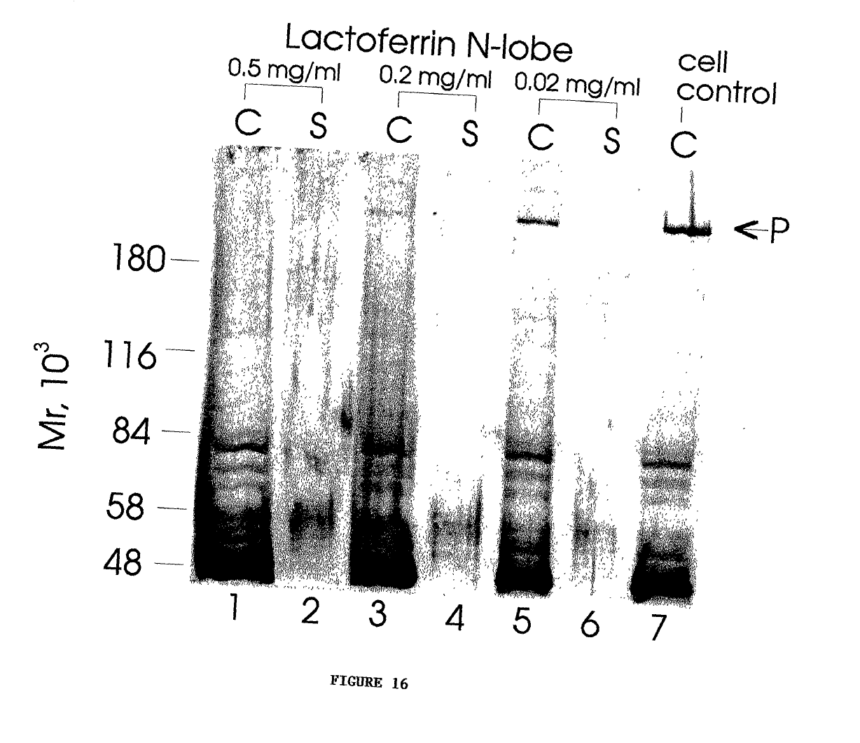

Comparison of the amino acid sequences of human, mouse, goat, pig, camel, and buffalo lactoferrins reveals conservation of Ser259, Lys73, and Asp315 in all cases. Similarly, the acidic residues near Ser259 are either invariant (Glu264, Asp379, and Glu388) or mostly conserved (Asp265). In this context, it is noteworthy that we have demonstrated proteolytic activity associated with native bovine lactoferrin purified from bovine milk. In contrast, as shown in FIGS. 14 and 15, the denatured bovine lactoferrin present in infant formulas is devoid of proteolytic activity.

Use

Because lactoferrin and its fragments inhibit the colonizing ability of infectious agents, lactoferrin preparations have significant therapeutic potential. Pharmaceutical compositions including lactoferrin, or fragments of lactoferrin, can be formulated for administration by the gastrointestinal tract. Examples of formulations for oral administration include tablets, capsules, and liquid formulations. Oral formulations containing native lactoferrin can be especially valuable as supplements for infant formulas. Native lactoferrin or recombinant lactoferrin formulations will also be useful as food preservatives, for example, in meat products. When lactoferrin is administered orally, it can enter the bloodstream, e.g., via gastrointestinal absorption, and can thereby affect tissues remote from the site of administration.

Lactoferrin and its fragments can also be formulated as eye drops, as nasal sprays, or as any other formulation suitable for inhalation.

Since non-pasteurized lactoferrin and fragments of lactoferrin inactivate proteins necessary for colonization, while leaving bacterial viability relatively unchanged, lactoferrin and its fragments may also be used to produce attenuated vaccines. For example, bacteria may be contacted with lactoferrin under conditions sufficient to extract and/or degrade the proteins in the bacterial cell walls, and the attenuated bacteria may then be formulated into a vaccine. Methods for preparing vaccines are known in the art and can be found, for example, in Vaccines, G. Slorein and E. Martance eds., 2d ed. Saunders, Harcourt-Brace 1994.

There now follow particular examples of the inactivation of infectious agents according to the invention. These examples are provided for the purpose of illustrating the invention, and should not be construed as limiting.

### EXAMPLE 1

### Extraction of Protease from H. influenzae Rd in Milk Whey

Haemophilus influenzae strain Rd is a nonencapsulated derivative of a serotype Rd strain that secretes type 1 IgA1 protease. The Rd strain was grown in brain heart-infusion broth supplemented with hemin (10 μg/ml) and nicotinamide adenine dinucleotide (2 μg/ml) to mid-log phase, and then harvested by centrifugation.

Human milk was obtained 3 to 6 days postpartum from healthy mothers taking no antibiotics, as described in Plaut et al., J. Infect. Dis. 166:43 (1992). All samples were collected in sterile beakers; within 6 hours of collection they were centrifuged at 10,000×g for 20 min at 4 C to remove lipids and cells. The resulting whey was stored at −70 C and was prepared for use by thawing slowly, without further modifications. 2×10⁹ H. influenzae cells were resuspended in 1 ml of the unmodified human milk whey and incubated at 37 C with gentle mixing. Samples were removed at intervals between 2 minutes and 1 hour. Whole cells and supernatants were examined by Western immunoblot using antisera directed against all domains of the preprotein.

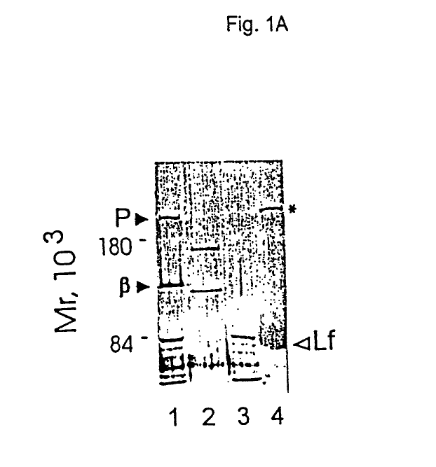

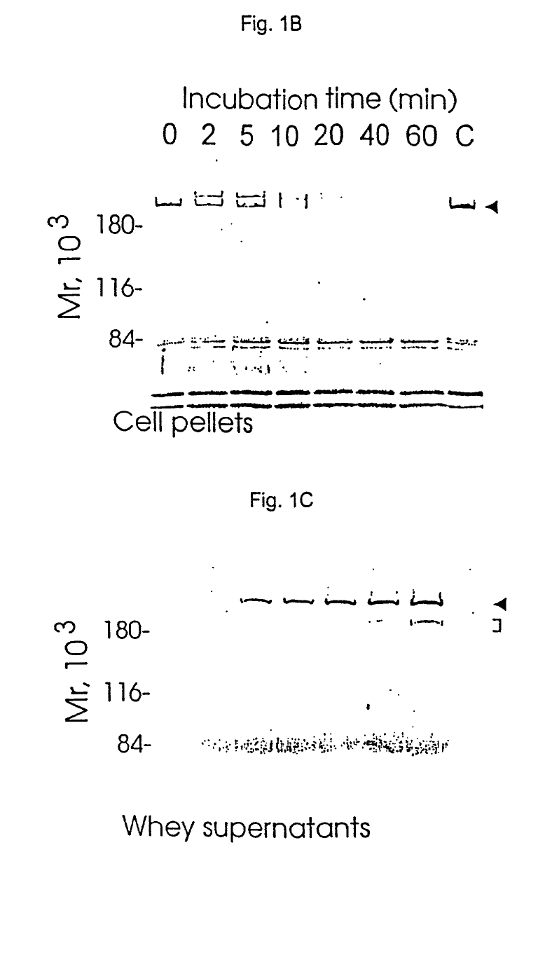

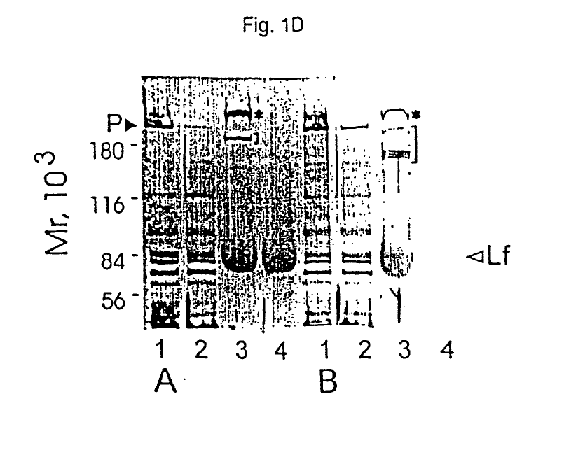

As shown in FIG. 1A, human milk whey removed the native IgA1 protease precursor and the remnant helper domain from wild-type Rd H. influenzae cells. Lanes 1 and 2 show broth cultures of Rd cells. The cells in Lane 1 contained preprotein (P), and the remnant helper domain (β) from processed preprotein. The broth supernatant in Lane 2 produced two main bands, both of which were active IgA1 proteases released during culture. Lane 3 shows the same Rd cells incubated 1 hour with milk whey, which removed the precursor and beta domains. The precursor (*) was transferred to the milk supernatant in Lane 4; it was unprocessed, since milk contains antibodies that inhibit processing of the precursor in solution. The extracted helper beta domain was unstable in solution, and was not detected. Lactoferrin is indicated by arrow Lf.

The antiserum used was anti-Rd3-13, which reacts with IgAp, IgA, and IgA. Lactoferrins (Lf) were detected by the second antibody, an enzyme-conjugated goat anti-rabbit IgG.

### EXAMPLE 2

### Extraction of Protease from H. influenzae RD3-13 in Milk Whey

H. influenzae strain Rd3-13 is an Rd derivative that expresses enzymatically inactive IgA1 protease which cannot autoprocess, leading to the accumulation of preprotein in the bacterial outer membrane. Mid-log phase Rd3-13 bacteria were incubated in milk whey, and aliquots were removed at the times shown. Just before extraction, the cell-associated preprotein ran at a higher than expected position on an electrophoretic gel.

Bacterial pellets (FIG. 1B) and their corresponding whey supernatants (FIG. 1C) were examined using unadsorbed rabbit anti-Rd3-13 preprotein. After incubation for 10 minutes in milk, only a small amount of IgA1 protease preprotein remained cell-associated; by 60 minutes, extraction was complete. Following transfer to the supernatant, the protein was very slowly degraded to lower-molecular-weight species. Solid arrowheads show the preprotein, and the brackets designate degradation products of the preprotein in whey. The controls, in lanes C, were Rd3-13 cells incubated for 60 minutes in buffer alone. Preprotein in the controls remained associated with the bacterial cells.

Quantitation of colony forming units of Rd or Rd3-13 after incubation in milk for two hours, when nearly all of the preprotein had been extracted, showed no effect on viability.

### EXAMPLE 3

### Determination of Active Constituents of Milk Whey

Milk whey proteins were fractionated by precipitating the proteins with acetone, then subjecting them to anion exchange (DE 52, Whatman, England), followed by molecular sieve chromatography (Biogel P 200, Pharmacia, Richmond, Calif.). All steps were performed in neutral buffers, at room temperature or 4 C in neutral buffers. The resulting fractions were tested for activity. In experiments with both Rd and Rd3-13, only fractions containing lactoferrin reproduced the findings with unmodified milk whey.

Highly purified recombinant forms of the full-length protein from two sources, baby hamster kidney (BHK) cells, and Aspergillus awamori recombinant lactoferrin (provided by Agennix Corporation, and described in Stowell et al., Biochem. J. 276, 349-355 (1991)), as well as the wild-type N-lobe of human lactoferrin produced in BHK cells, were tested.

Recombinant proteins were used at a concentration of 1 mg/ml (13 μM), approximating levels of lactoferrin in human milk (Masson et al., Clin. Chim. Acta 14, 735 (1966)). The results are shown in FIG. 1D. Lanes A1-4 are unmodified human milk whey; Lanes B1-4 are baby hamster kidney recombinant human lactoferrin. Lanes A1 and B1 show Rd3-13 cells (the arrow P shows the preprotein). Lanes A2 and B2 show cells after incubation in whey (A) or 13 μM recombinant lactoferrin (B); Lanes A3 and B3 show the corresponding supernatants. Lanes A4 and B4 show milk and lactoferrin controls containing no bacteria. The antiserum used was anti-Rd3-13, which reacts with IgAp, IgAa, and IgAb. Lactoferrins (Lf) were detected by the second antibody, an enzyme-conjugated goat anti-rabbit IgG.

As shown in FIG. 1D, lactoferrin purified from BHK cells removed the IgA1 protease preprotein (*) from strain Rd3-13, and then slowly degraded the extracted protein (Lanes B1-4, brackets). The N-lobe of human lactoferrin had an identical effect. In addition, both sources of lactoferrin caused an upward shift of the preprotein. Lactoferrin iron content, which was varied according to the protocol of Mazurier and Spik (Biochim. Biophys. Acta 629:399-408 (1980)), had no influence on either extraction or degradation.

To ensure that no other proteins were present in the recombinant lactoferrin preparations, molecular mass measurements of these proteins were carried out by mass spectroscopy using a Maldi-Tof linear instrument. The intact, glycosylated BHK lactoferrin was 79,338 daltons, and glycosylated N-lobe was 36,890 daltons. Both of these values were very close to the predicted values for these species.

### EXAMPLE 4

### Effect of Human Milk Lactoferrin on Hap Adhesin

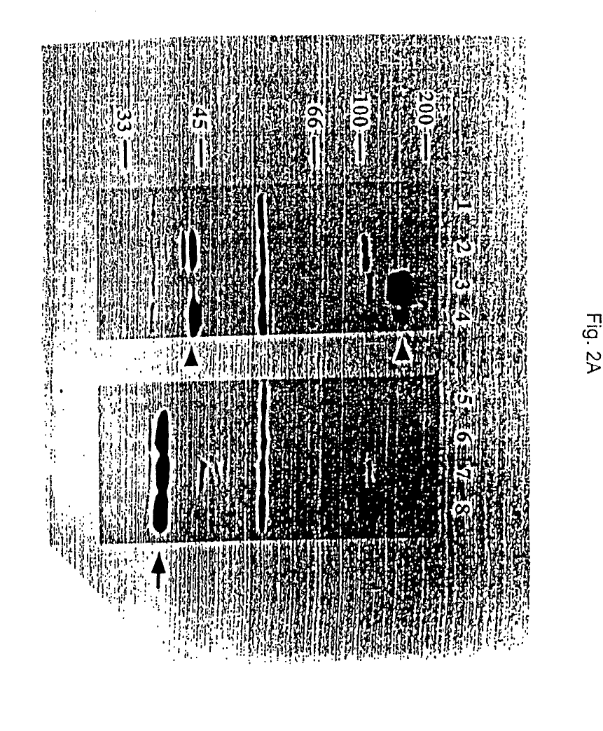

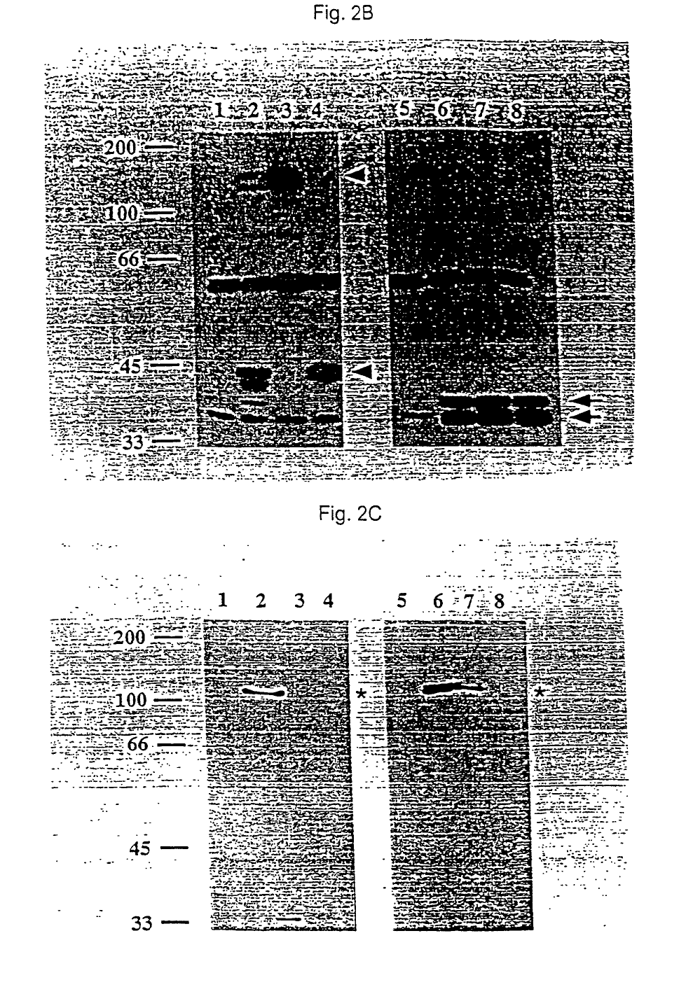

The effect of 13 μM human milk lactoferrin on the Hap adhesin, which is structurally similar to IgA1 protease, was also examined. FIG. 2A shows the analysis of whole cell lysates of H. influenzae strain DB117 derivatives preincubated with PBS alone (left), and with PBS and 13 μM human milk whey lactoferrin (right). FIG. 2B illustrates the analysis of whole cell lysates of H. influenzae strain DB117 derivatives preincubated with PBS alone (left), and with PBS and 13 μM A. awamori recombinant human lactoferrin (right). FIG. 2C shows the analysis of culture supernatants of H. influenzae strain DB117 derivatives preincubated with PBS alone (left), and with PBS and 13 μM A. awamori recombinant human lactoferrin (right).

Western analysis was performed with antiserum Rab730, which reacts with the Hap preprotein, Hap_{S}, and Hap. The gels in all panels were loaded as follows: Lane 1, DB117/vector with PBS; Lane 2, DB117/wild type Hap with PBS; Lane 3, DB117/HapS243A with PBS; Lane 4, DB117/Hap with PBS; lane 5, DB117/vector with lactoferrin; lane 6, DB117/wild type Hap with lactoferrin; Lane 7, DB117/HapS243A with lactoferrin; and Lane 8, DB117/Hap with lactoferrin. Arrowheads indicate the Hap preprotein and Hap, arrows indicate Hap degradation products, and asterisks indicate Hap_{S}.

As shown in FIGS. 2A-2C, lactoferrin treatment of strain DB117 expressing wild-type Hap resulted in proteolysis, rather than extraction of Hap. The preprotein and Hap were lost, and a C-terminal fragment slightly smaller than Hap (39 kDa vs. 45 kDa) appeared.

To determine whether proteolysis depended on Hap serine protease activity, the effect of lactoferrin on DB117 expressing Hap with a mutated active site serine (HapS243A) was examined. This protein lacks autoproteolytic activity and remains in the outer membrane in preprotein form. Western analysis of whole cells revealed loss of the Hap preprotein and generation of a Hap C-terminal fragment (FIG. 2A, Lanes 3 and 7). Treatment of DB117 expressing a Hap derivative containing the Hap signal sequence fused to Hap also resulted in generation of the cell-associated 39 kDa C-terminal fragment (FIG. 2A, Lanes 4 and 8), indicating that proteolysis of the exposed segment of Hapβ by lactoferrin could take place in the absence of the entire Hap_{S} domain.

### EXAMPLE 5

### Effect of Recombinant Human Lactoferrin on Hap

As shown in FIG. 2B, 13 μM recombinant human lactoferrin prepared from A. awamori generated two products, one being the same 39 kDa C-terminal fragment observed with milk-derived lactoferrin, and the other being a slightly smaller C-terminal fragment. Further analysis revealed that Hap_{S} or a related fragment of the Hap preprotein, was liberated into the supernatant (FIG. 2C).

Experiments comparing the proteolysis of Hap by 87 nM, 217 nM, 430 nM naturally-occurring human lactoferrin with 13 μM recombinant lactoferrin established a dose-response relationship, with proteolysis detectable but incomplete after treatment of cells for 1 hour with the lowest concentration.

Additional studies with BHK recombinant human lactoferrin yielded results that paralleled those obtained with A. awamori recombinant protein. As seen with IgA1 protease, the recombinant N-lobe behaved exactly like the full-length protein.

### EXAMPLE 6

### Inhibition of Hap-Mediated Attachment

Strain DB117 expressing HapS243A was incubated for 1 hour in PBS alone, and in PBS with 13 μM lactoferrin. It was washed twice, and then inoculated onto a monolayer of Chang epithelial cells. Following incubation for 30 minutes, adherence was quantitated as described by St. Geme III et al., Proc. Natl. Acad. Sci. USA 90: 2875 (1993). Adherence is reported relative to DB117/HapS243A after incubation in PBS, which was normalized to 100%.

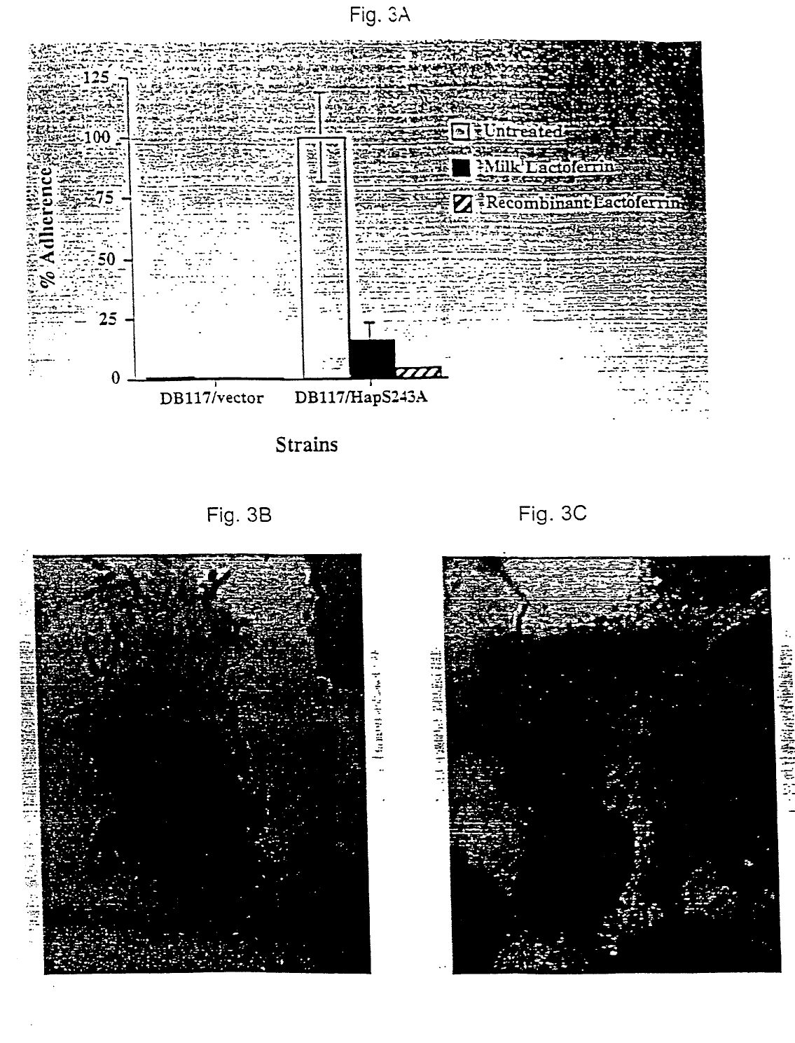

FIG. 3A illustrates adherence to Chang epithelial cells by DB117/vector and DB117/HapS243A after incubation in PBS, PBS with 13 μM human milk whey lactoferrin, or PBS with 13 μM A. awamori recombinant lactoferrin. FIGS. 3B and 3C show light micrographs of DB117/HapS243A associated with Chang epithelial cells samples after staining with Giemsa stain. The sample in FIG. 3B was incubated in PBS, and the sample in FIG. 3C was incubated with 13 μM A. awamori recombinant lactoferrin.

DB117 expressing HapS243A demonstrated augmented in vitro adherence compared with DB117 expressing wild-type Hap, reflecting the fact that attachment is mediated by the preprotein form of Hap, which remains intact and cell-associated when the active site serine is mutated. As shown in FIG. 3A, treatment of DB117/HapS243A with either milk-derived or recombinant lactoferrin resulted in an 85-97% decrease in Hap-mediated adherence. DB117/vector served as a negative control and was nonadherent, regardless of lactoferrin treatment.

### EXAMPLE 7

### Effect of Serine Protease Inhibitor PMSF on Lactoferrin-Associated Proteolysis of H. influenzae Hap

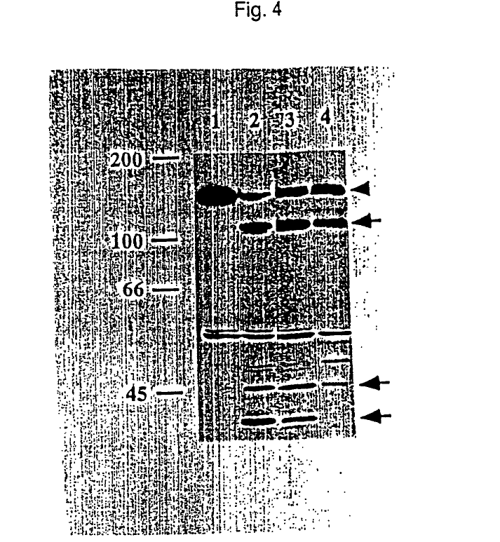

To determine whether lactoferrin was functioning as a serine protease, the ability of phenylmethylsulfonyl fluoride (PMSF), a broad inhibitor of serine proteases, to inhibit degradation of Hap was examined. The results are shown in FIG. 4. DB117/HapS243A was incubated in PBS (Lane 1), PBS with 430 nM A. awamori recombinant lactoferrin (Lane 2), PBS with 430 nM recombinant lactoferrin and 7.5% isopropanol (Lane 3), or PBS with recombinant lactoferrin and 7.5 mM PMSF in isopropanol (Lane 4). Whole cell lysates were prepared and examined by Western blot analysis with antiserum Rab 730, which reacts with the Hap preprotein, Hap_{S}, and Hap. The arrowhead indicates the Hap preprotein, and the arrows indicate Hap degradation products. As shown in FIG. 4, the partial proteolysis of Hap produced by 430 nM recombinant lactoferrin was significantly inhibited by 7.5 mM PMSF.

Lactoferrin extraction of the IgA1 protease preprotein was also inhibited in the presence of 10 mM PMSF or 10 mM diisopropylfluorophosphate (DFP, a second serine protease inhibitor).

### EXAMPLE 8

### Determination of the Specificity of the Interaction of Lactoferrin and H. influenzae Proteins

The H. influenzae major outer membrane proteins P2, P5, and P6 are predicted to form β-barrel structures that include a series of transmembrane antiparallel amphipathic sheets (Vachon et al., Biochim. Biophys. Acta 861: 74-82 (1986); Nelson et al., Infect. Immun. 56: 128-134 (1988); Deich et al., J. Bacteriol. 170: 489-498 (1988); Munson et al., Infect. Immun. 61: 4017-4020 (1993)), as do IgA and Hap. However, P2, P5, and P6 lack the characteristic large extracellular domains that link IgA and Hap to their N-terminal passenger domains in the autotransported proteins.

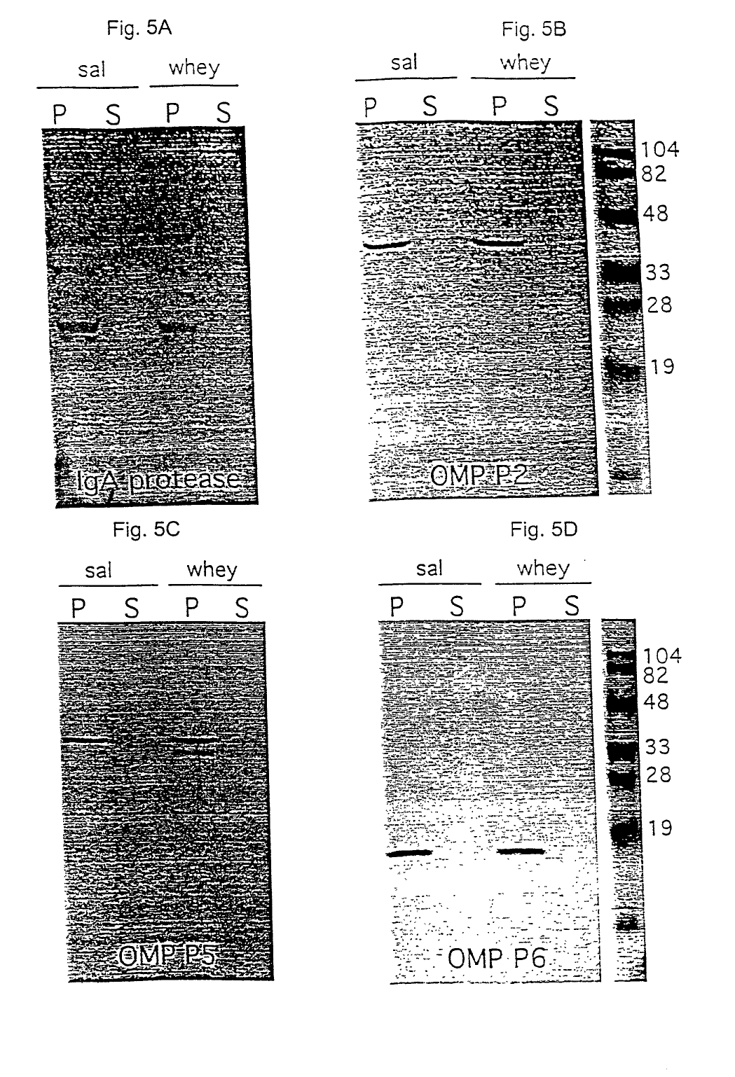

Logarithmic phase cells of H. influenzae were incubated with saline (sal) or whey; the results are shown in FIGS. 5A-5D. Cells were centrifuged, and the pellets (Lanes P) and corresponding supernatants (Lanes S) were examined by immunoblot assay. The panel marked IgA protease (FIG. 5A) was probed with rabbit serum #331, an antiserum that recognizes IgAp, IgA, and IgA. The other panels (FIGS. 5B-5D) were probed with monoclonal antibodies specific for the proteins noted: OMP P2: antibody 6G3; OMP P5: antibody 2C7; OMP P6: antibody 7F3. Proteins were detected with protein A peroxidase and horseradish peroxidase color developer. Cells in the OMP P2 panel (FIG. 5B) were strain 1479 for which antibody 6G3 is specific. Cells in all other panels were Rd3-13. Molecular mass markers (as kDa) are on the right.

As shown in FIGS. 5A-5D, the IgA protease precursor was translocated to supernatant from cells by milk whey, while the P2, P5, and P6 outer membrane proteins were unaffected. All three proteins remained cell associated.

### EXAMPLE 9

### Bacterial Strains and Plasmids

H. influenzae strain Rd is a capsule-deficient serotype d strain that secretes IgA1 protease but contains a nonfunctional hap gene because of a spontaneous nonsense mutation at codon 710 (Fleishmann, et al. (1995) Whole-genome random sequencing and assembly of Haemophilus influenzae Rd. Science 269, 496-512). Strain Rd3-13 is a derivative of Rd with a mutant IgA1 protease that lacks protease activity because of a valine in place of the active site serine at position 288 and remains cell-associated in the precursor form (Qiu et al., (1998) Human milk lactoferrin inactivates two putative colonization factors expressed by Haemophilus influenzae. Proc. Natl. Acad. Sci. USA 95, 12641-12646). Strain DB117 is a derivative of Rd with a mutation in the rec-1 gene and is deficient in recombination (Setlow et al., (1968) Repair of deoxyribonucleic acid in Haemophilus influenzae. I. X-ray sensitivity of ultraviolet-sensitive mutants and their behavior as hosts to ultraviolet-irradiated bacteriophage and transforming deoxyribonucleic acid. J. Bacteriol. 95, 546-558). E. coli DH5α is a laboratory strain that has been described previously (Sambrook et al., (1989) Molecular Cloning: A Laboratory Manual (Cold Spring Harbor, N.Y.: Cold Spring Harbor Laboratory Press)).

The plasmids pBR322, pGJB103, pUC19, and pNUT are cloning vectors that have been described previously (Palmiter et al., (1987) Cell lineage ablation in transgenic mice by cell-specific expression of a toxin gene. Cell 50, 435-443; Sambrook et al., 1989; Tomb et al., (1989). Transposon mutagenesis, characterization, and cloning of transformation genes of Haemophilus influenzae Rd. J. Bacteriol. 171, 3796-3802). pFG26 is a derivative of pBR322 and encodes wild type IgA1 protease (Grundy et al., (1987) Haemophilus influenzae immunoglobulin A1 protease genes: cloning by plasmid integration-excision, comparative analysis, and localization of secretion determinants. J. Bacteriol. 169, 4442-4450). pYFI-65 encodes IgA1 protease with a valine in place of the active site serine, resulting in a protease that lacks activity and is locked in the precursor form. pJS106 is a derivative of pGJB103 and encodes wild type Hap (St. Geme et al., (1994) A Haemophilus influenzae IgA protease-like protein promotes intimate interaction with human epithelial cells. Mol. Microbiol. 14, 217-233). pHapS243A is a derivative of pJS106 and encodes Hap with a mutation at the active site serine (HapS243A), resulting in a protein that lacks proteolytic activity and remains cell-associated in the precursor form (Hendrixson et al., (1997) Structural determinants of processing and secretion of the Haemophilus influenzae Hap protein. Mol. Microbiol. 26, 505-518).

Using recombinant PCR, epitope tags consisting of either 6[His] or RGS-6[His] were introduced into IgA1 protease in place of short stretches of the native protein encoded by either pFG26 (epitope D) or pYF1-65 (epitopes C, E, and F) (see FIG. 2). The resulting plasmids were linearized and then transformed into either strain Rd3-13 (epitope D) or strain Rd (epitopes C, E, and F), generating derivatives that were identified by screening for the acquisition (epitope D) or loss (epitopes C, E, and F) of IgA1 protease activity, as described previously (Grundy et al., (1990) Localization of the cleavage site specificity determinant of Haemophilus influenzae immunoglobulin A1 protease genes. Infect. Immun. 58, 320-331).

H. influenzae strains were grown as described previously, using tetracycline at a concentration of 5 μg/mL, as appropriate (St. Geme and Falkow, (1990) Haemophilus influenzae adheres to and enters cultured human epithelial cells. Infect. Immun. 58, 4036-4044). These strains were stored at −80° C. in brain heart infusion broth with 20% glycerol. E. coli strains were grown on Luria-Bertani agar or in LB broth, using tetracycline at a concentration of 12.5 μg/mL and ampicillin at a concentration of 100 μg/mL, as appropriate. E. coli strains were stored at −80° C. in LB broth with 50% glycerol.

### EXAMPLE 10

### Construction of N-lobe Lactoferrin Mutants

Lactoferrin amino acids are numbered beginning with the first residue in the mature protein (lacking the signal peptide). The plasmid pNUT::N-Lf(S259A) encodes a mutant N-lobe lactoferrin (N-Lf) with an alanine in place of the serine at position 259. This plasmid was constructed using recombinant PCR with BamHI and NotI restriction sites at the 5′ end and PstI and NotI restriction sites at the 3′ end of the PCR product. The resulting product was digested with BamHI and PstI and cloned into BamHI-PstI-digested pUC19, creating pUC19::N-Lf(S259A). After nucleotide sequencing to confirm the presence of the proper mutation and wild type flanking sequence, the insert was liberated by digestion with NotI, then cloned into NotI-digested pNUT to create pNUT::N-Lf(S259A).

The plasmid pNUT::N-Lf(K73A) encodes a mutant N-lobe lactoferrin with an alanine in place of the lysine at position 73, and pNUT::N-Lf(D315A) encodes a mutant N-lobe lactoferrin with an alanine in place of the aspartic acid at position 315. Both pNUT::N-Lf(K73A) and pNUT::N-Lf(D315A) were constructed using a scheme analogous to that described for pNUT::N-Lf(S259A).

The plasmid pNUT::N-Lf(triple mutant) encodes a mutant N-lobe lactoferrin with an alanine in place of the serine at position 259, an alanine in place of the lysine at position 73, and an alanine in place of the aspartic acid at position 315. This construct was generated by starting with pUC19::N-Lf(S259A) and first introducing an alanine in place of Lys73 and then an alanine in place of Asp315. The resulting insert was liberated by digestion with NotI, then cloned into NotI-digested pNUT.

The plasmid pNUT::N-Lf(P251V) was constructed in earlier work using M13 phage and oligonucleotide-directed mutagenesis (Nicholson et al., (1997) Mutagenesis of the histidine ligand in human lactoferrin: iron binding properties and crystal structure of the histidine-253 → methionine mutant. Biochemistry 36, 341-346.

### EXAMPLE 11

### Purification of Lactoferrin Derivatives

To purify recombinant wild type N-lobe lactoferrin, N-Lf(S259A), N-Lf(K73A), N-Lf(D315A), N-Lf(triple mutant), and N-Lf(P25 1V), pNUT derivatives were transfected into BHK cells. Expression was induced by the addition of 80 μM ZnSO₄, and the recombinant proteins were recovered from culture medium by ion-exchange chromatography, as described previously (Day et al., (1992) Studies of the N-terminal half of human lactoferrin produced from the cloned cDNA demonstrate that interlobe interactions modulate iron release. J. Biol. Chem. 267, 13857-13862). All of the proteins were purified in parallel using identical procedures and conditions, with the exception of N-Lf(P251V), which was available from an earlier preparation. Milk lactoferrin was purified from human milk whey using anion exchange and molecular sieve chromatography, as described previously (Qiu et al., 1998), and was then adjusted to a final concentration of 1 mg/mL in Tris buffered saline, pH 7.9.

### EXAMPLE 12

### Lactoferrin Treatment of Whole Bacteria and Immunoblot Analysis

To determine the effects of wild type N-lobe lactoferrin and mutant N-lobe lactoferrin on proteolysis of H. influenzae IgA1 protease and Hap, bacteria were grown to mid-log phase and washed once with phosphate buffered saline (PBS). Subsequently, 400 μL volumes were pelleted and resuspended in 150 μL of either PBS alone or PBS containing 1.35 μM of the appropriate N-lobe lactoferrin preparation. Next, bacteria were incubated for 1 hour at 37° C., then washed once with PBS and resuspended in either Laemmli buffer for analysis by Western blot or PBS for analysis of adherence.

Proteins were resolved by SDS-PAGE on 7-10% polyacrylamide gels, and Western blots were performed as previously described (Laemmli, U. K. (1970) Cleavage of structural proteins during the assembly of the head of bacteriophage. Nature 227, 680-685; Towbin et al., (1979) Electrophoretic transfer of proteins from polyacrylamide gels to nitrocellulose sheets: procedure and some applications. Proc. Natl. Acad. Sci. USA 76, 4350-4354). IgA1 protease was detected using rabbit antiserum #331, which reacts with full-length IgA1 protease and Igaₚ (the secreted passenger domain) (Plaut et al., (1992) Growth of Haemophilus influenzae in human milk: synthesis, distribution, and activity of IgA protease as determined by study of iga+ and mutant iga− cells. J. Infect. Dis. 166, 43-52). Hap was detected using guinea pig antiserum GP74, which was raised against purified Hap_{S} and reacts with full-length Hap and Hap_{S}, or with rabbit antiserum Rab730, which reacts with full-length Hap, Hap_{S}, and Hap_{β} (Hendrixson et al., 1997). The 6[His] epitope tag was detected with monoclonal antibody anti-RGS.6[His] (Qiagen, Valencia, Calif.).

### EXAMPLE 13

### Lys—C and Trypsin Digestion and MALDI Mass Spectrometry Analysis

To define the C-terminus of the IgA1 protease and Hap species released from the surface of the organism by lactoferrin, culture supernatants were resolved by SDS-PAGE, then stained with Coomassie blue R250. The appropriate band was excised from the gel and then washed twice with 200 μL 0.05 M Tris, pH 8.5/50% acetonitrile. After removing the washes, the gel pieces were dried for 30 minutes in a speed-vac and then digested overnight at 32° C. with either 0.1 μg of Lys—C or 0.05 μg of modified trypsin in 0.025 M Tris, pH 8.5. The resulting digests were dried again and then dissolved in 3 mL of matrix solution (10 mg/mL of 4-hydroxy-α-cyanocinnamic acid in 50% acetonitrile/0.1% TFA) for analysis by MALDI mass spectrometry.

Digests involving lactoferrin and synthetic peptides were extracted twice with 50 μL acetonitrile/2% TFA and then dried and resuspended in matrix solution for analysis by MALDI mass spectrometry.

MALDI mass spectrometric analysis was performed by the Protein Chemistry Core Facility of the Howard Hughes Medical Institute, Columbia University using a PerSeptive Voyager DE-RP mass spectrometer in the linear mode or by the Protein Chemistry Laboratory at Tufts University using a PerSeptive Biosystems Voyager Benchtop mass spectrometer in the linear mode.

### EXAMPLE 14

### Comparison of Lactoferrin Preparation of the Invention with Commercially Available Lactoferrin Preparations

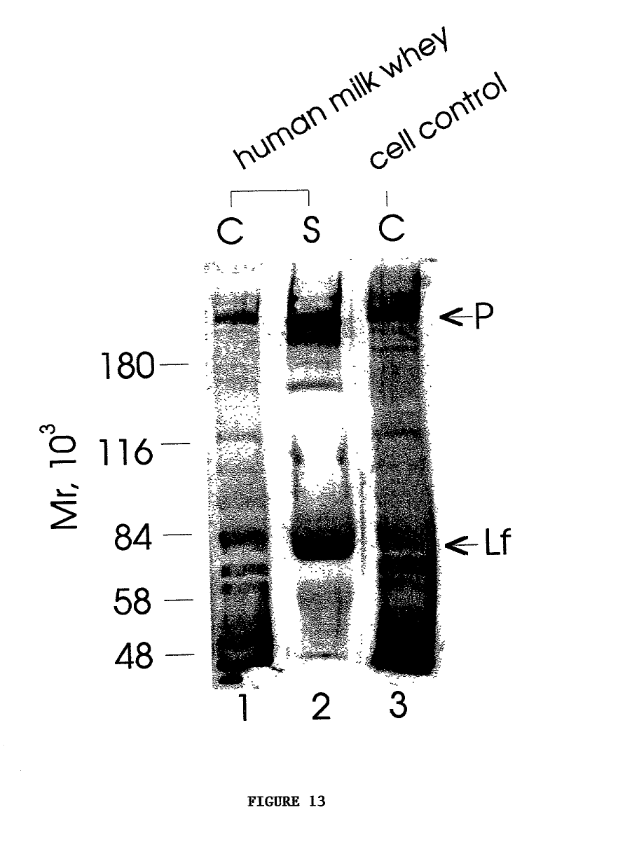

To demonstrate the difference in proteolytic activity of non-pasteurized lactoferrin of the present invention, as compared to commercially available lactoferrin, various lactoferrin preparations were analyzed for the ability to cleave and release the H. influenzae IgA protease precursor. The experimental conditions are identical to those described in Example 1, above. The results are depicted in FIGS. 13-16, which show Western blot analyses of 7% SDS-PAGE gels. In each figure, the primary antibody is polyclonal rabbit #331 anti-H. influenzae IgA protease, and the secondary antibody is goat anti-rabbit serum conjugated with alkaline phosphatase. The H. influenzae strain used was designated Rd3-13, a derivative of strain Rd in which the IgA protease precursor has been mutated to prevent its autocatalytic processing, thus ensuring that the full length protein (arrow P) remains intact on the bacterial outer membrane.

FIG. 13 demonstrates the proteolytic activity of a non-pasteurized lactoferrin of the invention. In particular, FIG. 13 demonstrates the removal of H. influenzae IgA protease precursor (arrow P) after 30 min. incubation with non-pasteurized lactoferrin from human milk whey. Lane 1 (labeled “C”) shows the results from the bacterial cell pellet, and reveals a small amount of precursor protein remaining. Lane 2 (labeled “S”) shows the results from the digest supernatant, and reveals large amounts of precursor protein and its degradation products. Lane 3 shows the results from the control cell, which was not incubated.

Commercially available bovine milk lactoferrin powders were used as supplied and dissolved in 20 mM Tris-buffered saline, pH 7.5 to a concentration of 1 mg/mL and for use in the assays. Unless otherwise indicated, the figures illustrate the result when the highest (1 mg/mL) concentrations were used to cleave the IgA protease precursor on H. influenzae Rd3-13. Incubation periods were 1 hr at 37° C., unless otherwise indicated.

“DMV Lactoferrin” was obtained from DMV International Nutritionals (Fraser, N.Y.; Lactoferrin Lot 10022340). The following compositional information was obtained from the supplier's website (http://www.lfplus.com/sl/13.html): “The separation technique used by DMV isolates the lactoferrin in its natural form, with its wide spectrum of nutritional properties. Thanks to a mild treatment process, the bioactivity of DMV Lactoferrin is high. The patented manufacturing process allows the isolation of lactoferrin from [bovine] milk or a milk derivative by ion exchange. The eluate is then filtered, dried, and packaged in a sealed fiberdrum with an aluminum laminated bag and a net content of 5 kg.” The following information was obtained from another page of the supplier's website (http://www.lfplus.com/sl/14.html): “In the original sealed packaging, DMV Lactoferrin has a shelf life of at least three years from production date, if stored below 20° C. This shelf life is applicable in the unopened packaging stored at moderate (max 75%) relative humidity. Lactoferrin is manufactured according to the Dairy Hygiene Directive 92/46 of the EC and meets the corresponding requirements. For the allowed use, the relevant food standards should be checked in your local geographic area, to determine permitted use.”

TABLE 4
Specifications and typical analysis of DMV Lactoferrin

| | Specification | Typical |
|---|---|---|
| Protein (N × 6.38) | min. 93.0% | 95.5% |
| Moisture | max. 4.5% | 4.0% |
| LF on Protein | min. 95.0% | 97.0% |
| Ash (550° C.) | max. 1.0% | 0.5% |
| pH (2%, 20° C.) | 5.2-6.2 | 5.7 |
| Bulk density | | 0.3 g/ml |
| Solubility: in water, 20° C. | | Completely at 2% |
| Solubility: transmittance, 2% sol, | min. 80% | 82% |
| 600 nm | | |
| Iron binding: spectrophotometric | min. 70% | 76% |
| method at 465 nm (on solids) | | |
| Foreign matter (10 g) | | Absent |
| Heavy metals (as lead) | max. 1 mg/kg | <0.25 mg/kg |
| Standard plate count | max. 1,000/g | 500/g |
| Enterobacteriaceae | max. 10/g | <10/g |
| Yeast | max. 10/g | <10/g |
| Moulds | max. 10/g | <10/g |
| Staphyloccocus aureus 2 × 1 g | negative | Negative |
| Salmonella | neg. in 50 g | neg. in 50 g |

Analytical data refers to internationally accepted methods (IDF, ISO, AOAC) and are available on special request.
Nutritional value for DMV Lactoferrin (per 100 g product): protein 95.5 g; moisture 4.0 g; ash 0.5 g.

FIG. 14 shows a comparison of proteolytic activity between non-pasteurized human milk lactoferrin and bovine milk lactoferrin (“DMV Lactoferrin”) from a commercial source. The incubation conditions with H. influenzae IgA protease precursor are the same as above. As shown in lane 3, after 15 minutes, a large amount of precursor has been proteolytically removed by the non-pasteurized human milk lactoferrin of the invention. In contrast, lane 7 shows that virtually no precursor was removed by the commercially available DMV Lactoferrin after 15 minutes. After two hours, only a trace of the precursor was removed by the commercially available DMV Lactoferrin (compare lane 11 with lanes 3 and 5).

Other commercially available bovine milk lactoferrin was obtained from Ecological Formulas (Concord, Calif.; “Lactoferrin—Bioactive Glycoprotein”; 100 mg capsules; lot number: 004885). The product label for this preparation states: “Calcium carbonate 98 mgm per capsule; Lactoferrin (bovine) 100 mgm per capsule. Other ingredients: Magnesium Stearate, Silicone dioxide. Individually resolved from whey protein. Each lot of lactoferrin has been tested for purity and potency. Specific separation techniques are used to isolate lactoferrin in its natural form . . . .”

The third preparation of commercially available bovine milk lactoferrin was obtained from Morinaga Milk Industry Co, Ltd. This product was distributed in five gram packets by Morinaga representatives at the Fifth International Conference on Lactoferrin: Structure, Function, and Applications, in Banff, Canada, May 4-9, 2001. The method for producing this preparation is disclosed in Bellamy et al., “Identification of the bactericidal domain of lactoferrin,” Biochim Biophys. Acta. May 22, 1992; 1121(1-2): 130-6.

FIG. 15 shows a comparison of the three commercially available bovine milk lactoferrin preparations discussed above. For this experiment, the control H. influenzae cell was incubated with buffer alone. After 1 hour, the only detectable activity was found in lane 9 (arrow P); however, it is clear that the amount of precursor removed after a 1 hour incubation is far less than that removed by the non-pasteurized lactoferrin of the present invention after only 30 minutes (see lane 2, FIG. 13).

FIG. 16 shows the dose-dependent enzymatic activity of recombinant human lactoferrin N-lobe expressed in baby hamster kidney cells. Three different concentrations (0.5 mg/mL, 0.2 mg/mL, and 0.02 mg/mL) of recombinant human lactoferrin N-lobe were incubated with H. influenzae IgA protease precursor for 30 minutes. The two highest concentrations of N-lobe markedly depleted the precursor (arrow P) from the cell surface (lanes 1 and 3). The protein removed is not seen in the supernatant (lanes 2 and 4), due to the extensive secondary proteolytic degradation by the recombinant N-lobe. These results demonstrate the potent proteolytic activity found in the recombinant N-lobe alone. Low levels of recombinant N-lobe (lanes 5 and 6) did not cleave the substrate.

Peptide Synthesis

Synthetic peptides were generated by the Protein Chemistry Laboratory at Tufts University using f-moc chemistry with HBTU activation (Applied Biosystems 431A).

Adherence Assays

Adherence assays were performed with A549 respiratory epithelial cells (ATCC CCL 185), as previously described (St. Geme et al., (1993) High-molecular-weight proteins of nontypable Haemophilus influenzae mediate attachment to human epithelial cells. Proc. Natl. Acad. Sci. USA 90, 2875-2879). A549 cells were maintained in F-12 medium with 10% heat-inactivated fetal calf serum. Percent adherence was calculated by dividing the number of adherent colony-forming units per monolayer by the number of inoculated colony-forming units.

All publications and patents mentioned in this specification are herein incorporated by reference to the same extent as if each individual publication or patent was specifically and individually indicated to be incorporated by reference.

Other Embodiments

From the foregoing description, it will be apparent that variations and modifications may be made to the invention described herein to adopt it to various usages and conditions. Such embodiments are also within the scope of the following claims.

What is claimed is:

1. A method for substantially reducing the pathogenicity of an infectious agent, without killing said infectious agent, by removing or degrading a surface protein of said infectious agent, said method comprising contacting said infectious agent with substantially pure, non-pasteurized, naturally occurring lactoferrin under conditions sufficient to remove or degrade said protein.

2. The method of claim 1, wherein said infectious agent is a bacterium.

3. The method of claim 1, wherein said infectious agent is a virus.

4. The method of claim 1, wherein said infectious agent is H. influenzae.

5. The method of claim 1, wherein said protein is an autotransported colonization factor.

6. The method of claim 1, wherein said protein is IgA1 protease.

7. The method of claim 1, wherein said protein is an adhesin.

8. The method of claim 1, wherein said protein is Hap.

9. A method for substantially reducing the pathogenicity of an infectious agent, without killing said infectious agent, by removing or degrading a surface protein of said infectious agent, said method comprising contacting said infectious agent with recombinant lactoferrin under conditions sufficient to remove or degrade said protein.

10. A method for substantially reducing the pathogenicity of an infectious agent, without killing said infectious agent, by removing or degrading a surface protein of said infectious agent, said method comprising contacting said infectious agent with a substantially pure fragment of non-pasteurized, naturally occurring lactoferrin under conditions sufficient to remove or degrade said protein.

11. The method of claim 10, wherein said fragment is the N-terminal lobe of lactoferrin.

12. A method of inhibiting microbial colonization in a mammal comprising administering to said mammal a therapeutically effective amount of substantially pure, non-pasteurized, naturally-occurring lactoferrin.

13. The method of claim 12, wherein said mammal is a human.

14. A method of inhibiting microbial colonization in a mammal comprising administering to said mammal a therapeutically effective amount of a substantially pure fragment of non-pasteurized, naturally-occurring lactoferrin.

15. The method of claim 14, wherein said fragment is the N-terminal lobe of lactoferrin.

16. A method for substantially inactivating an infectious agent comprising contacting said infectious agent with substantially pure, non-pasteurized, naturally-occurring lactoferrin under inactivating conditions.

17. A method for substantially inactivating an infectious agent comprising contacting said infectious agent with a substantially pure fragment of lactoferrin under inactivating conditions, wherein said fragment has at least 100 amino acid residues.

18. The method of claim 17, wherein said fragment has at least 200 amino acid residues.

19. The method of claim 17, wherein said fragment is the N-terminal lobe of lactoferrin.

20. The method of claim 17, wherein said fragment is non-pasteurized.

21. The method of claim 17, wherein said fragment is isolated from naturally-occurring lactoferrin.

22. An antimicrobial pharmaceutical composition comprising substantially pure, non-pasteurized, naturally-occurring lactoferrin and a pharmaceutically acceptable carrier.

23. The composition of claim 22, wherein said composition is formulated for administration by the gastrointestinal tract, by inhalation, by the mucous membranes, or by the eyes.

24. The composition of claim 22, wherein said composition is formulated for oral administration.

25. An antimicrobial pharmaceutical composition comprising a substantially pure fragment of non-pasteurized, naturally-occurring lactoferrin and a pharmaceutically acceptable carrier.

26. The composition of claim 25, wherein said fragment is the N-terminal lobe of lactoferrin.

27. A method for producing an attenuated vaccine comprising the steps of
(a) contacting an infectious agent with lactoferrin under conditions sufficient to substantially inactivate said infectious agent; and
(b) formulating said inactivated infectious agent into a vaccine.

28. The method of claim 27, wherein said lactoferrin is non-pasteurized.

29. The method of claim 27, wherein said lactoferrin is isolated from a naturally-occurring source.

30. A method for producing an attenuated vaccine comprising the steps of
(a) contacting an infectious agent with a substantially pure fragment of lactoferrin under conditions sufficient to substantially inactivate said infectious agent; and
(b) formulating said inactivated infectious agent into a vaccine.

31. The method of claim 30, wherein said fragment is the N-terminal lobe of lactoferrin.

32. An attenuated vaccine comprising a substantially inactivated infectious agent, wherein said infectious agent is inactivated with lactoferrin.

33. The vaccine of claim 32, wherein said lactoferrin is non-pasteurized.

34. The vaccine of claim 32, wherein said lactoferrin is isolated from a naturally-occurring source.

35. An attenuated vaccine comprising a substantially inactivated infectious agent, wherein said infectious agent is inactivated with a substantially pure fragment of lactoferrin.

36. The vaccine of claim 35, wherein said fragment is the N-terminal lobe of lactoferrin.

37. A substantially pure peptide consisting of the N-terminal lobe of lactoferrin, wherein said lobe is isolated from non-pasteurized, naturally-occurring lactoferrin.
